(19) United States
(12) Patent Application Publication
(10) Pub. No.: US 2026/0144793 A1
(43) Pub. Date: May 28, 2026

(54) METHODS OF TREATING CANCER

(71) Applicants: AMGEN INC., Thousand Oaks, CA (US); Revolution Medicines, Inc., Redwood City, CA (US)

(72) Inventors: Haby Henary, Moorpark, CA (US); Alison Rebecca Meloni, Thousand Oaks, CA (US); Neelesh Soman, Oak Park, CA (US); Mark A. Goldsmith, Redwood City, CA (US); Steve Kelsey, Redwood City, CA (US); Robert J. Nichols, Redwood City, CA (US); Christopher J. Schulze, Redwood City, CA (US); Mallika Singh, Redwood City, CA (US); Jacqueline Smith, Redwood City, CA (US); Sophia Aparna Sohoni, Redwood City, CA (US); Xiaolin Wang, Redwood City, CA (US); Zhengping Wang, Redwood City, CA (US); David E. Wildes, Redwood City, CA (US)

(21) Appl. No.: 18/880,867

(22) PCT Filed: Jul. 11, 2023
(86) PCT No.: PCT/US23/27369

§ 371 Date: Jan. 3, 2025

Related U.S. Application Data
(60) Provisional application No. 63/388,098, filed on Jul. 11, 2022.

(51) Int. Cl. A61K 31/519; A61K 31/497; A61P 35/00

(52) U.S. Cl. CPC A61K 31/519; A61K 31/497; A61P 35/00

(57) ABSTRACT

Provided herein are methods of treating a cancer in a patient wherein the patient has a KRAS G12C mutation, comprising administering to the patient a therapeutically effective combination of sotorasib and RMC-4630.

### CROSS-REFERENCE TO RELATED APPLICATION

This application claims the benefit of priority to U.S. Provisional Patent Application No. 63/388,098, filed Jul. 11, 2022, which is hereby incorporated by reference in its entirety for all purposes.

### BACKGROUND

Kirsten rat sarcoma (KRAS) G12C mutation has been identified as a putative oncogenic driver in several types of solid tumors including non-small cell lung cancer (NSCLC) (Fernández-Medarde and Santos, 2011), colorectal cancer (CRC) (Jones et al, 2017), and other solid tumors such as pancreatic, endometrial, bladder, ovarian, and small cell lung cancer (The AACR Project GENIE Consortium, 2017; Zhou et al, 2016). Sotorasib, a KRAS^{G12C} inhibitor, has shown promising anti-tumor activity and favorable safety as monotherapy.

Tumor development in mutant KRAS driven murine models of NSCLC can be inhibited by genetic deletion of Ptpn11, the gene encoding Src homology region 2-containing protein tyrosine phosphatase 2 (SHP2) (Ruess et al, 2018). Src homology region 2-containing protein tyrosine phosphatase 2 (SHP2) is a non-receptor protein tyrosine phosphatase that regulates cell survival and proliferation through the Ras-extracellular signal-regulated kinase and other pathways. The compound {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol, also referred to as RMC-4630, is an investigational small molecule inhibitor of SHP2 that is being explored.

A need exists for combination therapies for KRAS mutant cancers.

### SUMMARY

Provided herein are methods of treating cancer in a patient, wherein the patient has a KRAS G12C mutation, the method comprising administering to the patient a therapeutically effective combination of sotorasib and {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol (RMC-4630).

### BRIEF DESCRIPTION OF THE DRAWINGS

FIG. 1 shows tumor response for patients having non-small cell lung cancer (NSCLC) administered 960 mg sotorasib and varying amounts of RMC-4630 (100 mg, 140 mg, or 200 mg), ordered by dosage amount of RMC-4630. KRAS^{G12C}-treatment naïve patients are noted with shading.

FIG. 2 shows the same data as FIG. 1 for tumor response for patients with NSCLC, but ordered by increasing response.

FIG. 3 shows treatment duration of the patients with NSCLC administered 960 mg sotorasib and varying amounts of RMC-4630 (100 mg, 140 mg, or 200 mg). The median follow up was 5.8 months across all patients. uPR noted on the graph indicated an unconfirmed partial response.

### DETAILED DESCRIPTION

While sotorasib monotherapy appears to drive substantial anti-tumor activity, combination therapies may be useful to achieve maximal efficacy and deeper and more durable responses. Non-clinical data demonstrates that the potency of KRAS G12C inhibitor treatment can be modulated by upstream receptor tyrosine kinase (RTK) activation (Lito et al, 2016) and concurrent inhibition of RTK signaling can lead to maximal KRAS G12C inhibition (Lito et al, 2016; Canon et al, 2019). The addition of a SHP2 inhibitor like RMC-4630 to a targeted therapy against mutant KRAS may further inhibit KRAS signaling, eliminate residual or bypass upstream RTK activity, and thereby enhance the anti-tumor activity.

Based on preclinical data, non-clinical toxicity studies, and the clinical experience with sotorasib and RMC-4630, the overall benefit/risk profile favors clinical development of this combination for patients with KRAS G12C-driven cancers. Based on the potential benefit and the protocol defined assessments for monitoring and mitigating any potential toxicities from the combination therapy (from either of the study drugs in combination or synergistic toxicity concerns from both), the benefit risk assessment supports the conduct of a clinical trial of sotorasib in combination with RMC-4630.

Thus, provided herein are methods of treating a cancer in a patient who has a KRAS G12C mutation, the method comprising administering to the patient a therapeutically effective combination of sotorasib and RMC-4630.

As used herein, an “effective amount,” “therapeutically effective” combination, or a “therapeutically effective” amount refers to an amount sufficient to elicit the desired biological response. In the present disclosure the desired biological response is to treat the cancer of the patient (i.e., a subject in need of treatment with both sotorasib and RMC-4630), such as slowing progression of the cancer, or reducing cancer presence in the patient. It is understood that reference to “a patient” in the present disclosure refers to a patient in need of treatment with a combination of sotorasib and RMC-4630. The precise amount of sotorasib and RMC-4630 administered to a patient will depend on the mode of administration, the type and severity of the cancer and on the characteristics of the subject, such as general health, age, sex, body weight and tolerance to drugs. In some embodiments, the effective amount of sotorasib and RMC-4630 of the combination is different from the effective amount of sotorasib and/or RMC-4630 administered as a monotherapy of either. In some embodiments, the effective amount of sotorasib and RMC-4630 of the combination is the same as the effective amount of sotorasib and RMC-4630 administered as a monotherapy of each. In some embodiments, the therapeutically effective combination of sotorasib and RMC-4630 refers to an amount of sotorasib and RMC-4630 that is different from the therapeutically effective amount of sotorasib and RMC-4630 administered as a monotherapy. In some embodiments, the therapeutically effective combination of sotorasib and RMC-4630 refers to an amount of sotorasib and RMC-4630 that is the same as the therapeutically effective amount of one of sotorasib and RMC-4630 administered as a monotherapy.

The sotorasib and RMC-4630 can be administered sequentially or simultaneously in separate formulations. Combination administration can encompass administration of the sotorasib and RMC-4630 in an essentially simultaneous manner, for example, in multiple, separate capsules or tablets for each. In addition, such combination administration can also encompass use of each of sotorasib and RMC-460 in a sequential manner in either order. When separately administered, the administration of sotorasib and RMC-4630 can be simultaneous (e.g., within about 5-10 minutes of each other), or separated by 1 or more hours (e.g., 1 hour, 2 hours, 3 hours, 4 hours, 5 hours, 6 hours, 7 hours, 8 hours, 9 hours, 10 hours, 11 hours, 12 hours, 24 hours, 26 hours, 48 hours, or 72 hours). In some cases, sotorasib is administered before RMC-4630. In some other cases, sotorasib is administered after RMC-4630.

Note that when a range or amount is provided in the disclosure herein, +/−5% of each range endpoint or specific amount is included, unless otherwise indicated. For example, a range of 50 to 200 mg RMC-4630 is understood to encompass 50+/−5% mg to 200+/−5% mg, e.g., 47.5 mg to 210 mg RMC-4630.

### Sotorasib

Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1M)-1-[4-methyl-2-(propan-2-yl) pyridin-3-yl]-4-[(2S)-2-methyl-4-(prop-2-enoyl)piperazin-1-yl]pyrido[2,3-d]pyrimidin-2 (1H)-one and has the following structure:

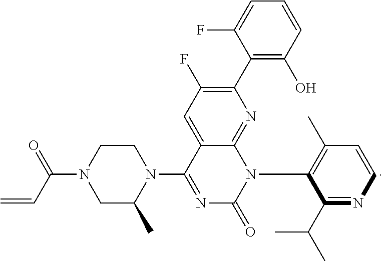

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019). See also, LUMAKRAS® US Prescribing Information, Amgen Inc., Thousand Oaks, California, 91320 (revision May 2021), which is herein incorporated by reference in its entirety.

In various embodiments, sotorasib is administered at a dose of 960 mg daily. In some embodiments, the sotorasib is administered once daily.

### RMC-4630

RMC-4630 is the compound {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol, disclosed in U.S. Pat. No. 10,590,090, and having a structure:

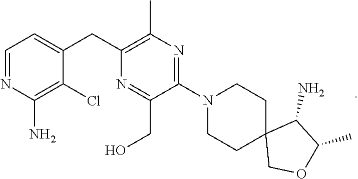 [SMILES: Cc1nc(N2CCC3(CC2)CO[C@@H](C)[C@H]3N)c(CO)nc1Cc1ccnc(N)c1Cl]

RMC-4630 and {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol are used interchangeably herein.

RMC-4630 is an orally bioavailable, potent, and selective SHP2 allosteric inhibitor that is being developed for patients with tumors harboring certain activating mutations or other genetic aberrations in the RAS-mitogen-activated protein kinase (MAPK) pathway, including upstream mutations in RTKs.

The pharmacology data demonstrate that RMC-4630 can inhibit RAS-MAPK pathway activation and growth in human cancer cells that bear distinct mutations in KRAS, such as KRAS G12C. RMC-4630 also inhibits the growth of human cancer cell lines that bear mutations, amplifications or activating rearrangements in RTKs, upstream of SHP2 in the RAS-MAPK signal transduction pathway.

Oral daily administration of RMC-4630 exhibited dose-dependent inhibition of tumor growth, and occasionally tumor regressions, in multiple solid tumor CDX models bearing the RAS-MAPK pathway activating mutations of interest; NSCLC NCI-H358 KRAS G12C, pancreatic ductal adenocarcinoma MIA PaCa-2 KRAS G12C, NSCLC NCI-H2122 KRAS G12C. Intermittent (e.g., every other day) dosing of RMC-4630 resulted in equivalent, or greater, anti-tumor activity in the NSCLC NCI-H358 KRAS G12C model and was better tolerated than corresponding daily dosing regimens.

RMC-4630 is currently being evaluated as a monotherapy in a first in human (FIH), dose-escalation, phase 1 study (RMC-4630-01; NCT03634982) in adult subjects with relapsed/refractory solid tumors. The purpose of this study is to evaluate safety, tolerability, preliminary efficacy, pharmacokinetics (PK), and pharmacodynamics (PD) of RMC-4630 monotherapy. The primary objective is to define the maximum tolerated dose (MTD) and the recommended phase 2 dose (RP2D) and schedule for RMC-4630 monotherapy.

In various embodiments, RMC-4630 is administered at a dose of 50 mg to 200 mg. In some embodiments, RMC-4630 is administered at a dose of 100 mg. In some embodiments, RMC-4630 is administered at a dose of 140 mg. In some embodiments, RMC-4630 is administered at a dose of 200 mg. In some embodiments, RMC-4630 is administered at a dose of 50 mg. In various embodiments, RMC-4630 is administered orally on days 1 and 2 of each 7 days (referred to as D1D2 herein). In some embodiments, RMC-4630 is administered orally on days 1 and 4 of each 7 days (referred to as D1D4 herein).

In various embodiments, RMC-4630 is administered when the patient is in a fasted state. A fasted state is when the patient has not eaten for at least 1 hour (e.g., at least 2 hours, at least 3 hours, at least 4 hours, or at least 8 hours) before and at least 1 hour (e.g., at least 2 hours, at least 3 hours, at least 4 hours, at least 8 hours) after administration of a drug. In various embodiments, the patient fasts for at least 2 hours before the administration of the RMC-4630. In some embodiments, the patient fasts for at least 1 hour after the administration of the RMC-4630.

### Patient Characteristics

The methods disclosed herein comprise treating a cancer in a patient who has a KRAS G12C mutation comprising administering to the patient a therapeutically effective combination of sotorasib and RMC-4630. Determination of a KRAS G12C mutation can be made as discussed in detail below.

In various embodiments, the patient exhibits an Eastern Cooperative Oncology Group (ECOG) performance status of 0 or 1 (see, e.g., Zubrod et al., 1960). Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare, and totally confined to bed or chair. Status 5 indicates death.

In various embodiments, the patient does not have a primary brain tumor. In various embodiments, the patient does not have spinal cord compression or untreated or symptomatic brain metastases or leptomeningeal disease from nonbrain tumors. In various embodiments, (i) the patient had brain metastases resected, or received whole brain radiation therapy ending at least 4 weeks prior to start of first treatment cycle, or received stereotactic radiosurgery ending at least 2 weeks prior to first treatment cycle, and (ii) the patient exhibits residual neurological symptoms of grade 2 or less, and receives a stable dose of dexamethasone, if applicable; and has an magnetic resonance imaging (MRI) performed within 30 days prior to first treatment cycle shows no new or enlarging lesions appearing. The phrase “active brain metastases” as used herein refers to a cancer that has spread from the original (primary) tumor to the brain. In some embodiments, a patient having active brain metastases has at least one measurable intracranial lesion >5 mm. In some embodiments, a patient having active brain metastases has at least one measurable intracranial lesion >5 mm but <10 mm. In some embodiments, a patient having active brain metastases has at least one measurable intracranial lesion >10 mm. See the National Cancer Institute Common Terminology Criteria for Adverse Events v5.0 (NCI CTCAE) published Nov. 27, 2017 by the National Cancer Institute, incorporated herein by reference in its entirety. See also Response Assessment in Neuro-Oncology Brain Metastases (RANO-BM) (Lin et al, 2015). RANO-BM is an extension of the Response Evaluation Criteria in Solid Tumors (RECIST) 1.1 (Eisenhauer et al, 2009) and the Response Assessment in Neuro Oncology (RANO) (Wen et al, 2010) response assessment criteria for high-grade gliomas.

In various embodiments, the patient does not have either systolic blood pressure of 160 mmHg or greater, or diastolic blood pressure of 100 mmHg or greater.

In various embodiments, the patient is in further need of treatment with a cytochrome P450 3A4 (CYP3A4) substrate. In some embodiments, the patient is not administered a CYP3A4 substrate in combination with sotorasib and RMC-4630. In various embodiments, the patient is not administered a CYP3A4 substrate during any treatment cycle or within 14 days or 5 half-lives of the CYP3A4 substrate or its major active metabolite, whichever is longer, before starting administration of sotorasib and RMC-4630. Exemplary CYP3A4 substrates include, but are not limited to, abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus (fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone. See, e.g., Flockhart D A, Drug Interactions: Cytochrome P450 Drug Interaction Table. Indiana University School of Medicine (2007), https://drug-interactions.medicine.iu.edu, accessed May 2021. In some embodiments, the CYP3A4 substrate is alfentanil, cyclosporine, dihydroergotamine, ergotamine, everolimus, fentanyl, primozide, quinidine, tacrolimus, or sirolimus. In some embodiments, the CYP3A4 substrate is a CYP3A4 substrate with a narrow therapeutic index. Exemplary CYP3A4 substrates with a narrow therapeutic index include, but are not limited to, alfentanil, cyclosporine, dihydroergotamine, ergotamine, everolimus, fentanyl, primozide, quinidine, tacrolimus, and sirolimus.

In various embodiments, the patient is in further need of treatment with a P-glycoprotein (P-gp) substrate. In some embodiments, the patient is not administered a P-gp substrate in combination with sotorasib and RMC-4630. In various embodiments, the patient is not administered a P-gp substrate during any treatment cycle or within 14 days or 5 half-lives of the P-gp substrate or its major active metabolite, whichever is longer, before starting administration of sotorasib and RMC-4630. Exemplary P-gp substrates include, but are not limited to, etexilate, digoxin, fexofenadine, everolimus, cyclosporine, sirolimus, tacrolimus, and vincristine. See, e.g., www.fda.gov/drugs/drug-interactions-labeling/drug-development-and-drug-interactions-table-substrates-inhibitors-and-inducers, accessed May 2021. In some embodiments, the P-gp substrate is etexilate, digoxin, fexofenadine, everolimus, cyclosporine, sirolimus, tacrolimus, or vincristine. In some embodiments, the P-gp substrate is a P-gp substrate with a narrow therapeutic index. Exemplary P-gp substrates with a narrow therapeutic index include, but are not limited to, digoxin, everolimus, cyclosporine, tacrolimus, sirolimus, and vincristine.

In various embodiments, the patient is in further need of treatment with a CYP3A4 inducer. In some embodiments, the patient is not administered a CYP3A4 inducer in combination with sotorasib and RMC-4630. In some embodiments, the patient is not administered a strong CYP3A4 inducer during any treatment cycle or within 14 days or 5 half-lives of the strong CYP3A4 inducer, whichever is longer, before starting administration of sotorasib and RMC-4630. Exemplary CYP3A4 inducers include, but are not limited to, apalutamide, avasimibe, barbiturate, brigatinib, carbamazepine, clobazam, dabrafenib, efavirenz, elagolix, enzalutamide, eslicarbazepine, glucocorticoids, ivosidenib, letermovir, lorlatinib, lumacaftor, mitotane, modafinil, nevirapine, oritavancin, oxcarbazepine, perampanel, phenobarbital, phenytoin, pioglitazone, rifabutin, rifampin, rifapentine, St. John's wort, telotristat, and troglitazone. See, e.g., Flockhart D A, Drug Interactions: Cytochrome P450 Drug Interaction Table. Indiana University School of Medicine (2007), www.drug-interactions.medicine.iu.edu, accessed May 2021. In some embodiments, the patient is not administered a strong CYP3A4 inducer in combination with sotorasib and RMC-4630. Exemplary strong CYP3A4 inducers include, but are not limited to, rifampin, mitotane, avasimibe, rifapentine, apalutamide, ivosidenib, phenytoin, carbamazepine, enzalutamide, St John's wort extract, and lumacaftor. See, e.g., www.fda.gov/drugs/drug-interactions-labeling/drug-development-and-drug-interactions-table-substrates-inhibitors-and-inducers, accessed May 2021. In some embodiments, the strong CYP3A4 inducer is rifampin, mitotane, avasimibe, rifapentine, apalutamide, phenytoin, carbamazepine, enzalutamide, ivosidenib, St. John's wort extract, or lumacaftor.

In some embodiments, the patient is further in need of treatment with a proton pump inhibitor (PPI) or an H2 receptor antagonist (H2RA). In some embodiments, the patient is not administered a PPI or a H2RA during any treatment cycle or within three days of starting administration of sotorasib and RMC-4630. Exemplary PPIs include, but are not limited to, omeprazole, pantoprazole, esomeprazole, lansoprazole, rabeprazole, or dexlansoprazole. Exemplary H2RAs include, but are not limited to, famotidine, ranitidine, cimetidine, nizatidine, roxatidine and lafutidine. In some embodiments, the patient, who is in further need of treatment with a PPI or H2RA, is not administered a proton pump inhibitor or a H2 receptor antagonist in combination with sotorasib and RMC-4630. In some embodiments, the PPI is lansoprazole, omeprazole, pantoprazole, raveprazole, or esomeprazole. In some embodiments, the H2RA is cimetidine, famotidine, nizatidine, or ranitidine.

In some embodiments, the patient is not administered a medication that prolongs QTc interval. Exemplary medications that prolong QTc interval include, but are not limited to, amiodarone, azithromycin, cilostazol, disopyramide, dronedarone, escitalopram, haloperidol, methadone, oxaliplatin, procainamide, sevoflurane, vandetanib, anagrelide, chloroquine, ciprofloxacin, dofetilide, droperidol, flecainide, ibutilide, moxifloxacin, pentamidine, propofol, sotalol, arsenic trioxide, chloropromazine, citalopram, donepezil, erythromycin, fluconazole, levofloxacin, ondansetron, pimozide, quinidine and thioridazine.

### Determination of KRAS G12C Mutation in Cancer

The patient treated in the methods disclosed herein is one suffering from a cancer who has a KRAS G12C mutation. In various embodiments, the patient has a cancer that was determined to have one or more cells expressing the KRAS G12C mutant protein prior to administration as disclosed herein. The presence or absence of KRAS G12C mutation in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences of wild-type human KRAS (nucleotide sequence set forth in Genbank Accession No. BC010502; amino acid sequence set forth in Genbank Accession No. AGC09594) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TaqMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including, but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one or more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; DeRisi et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), and a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is a fresh tumor or cancer sample. In some embodiments, the sample is a frozen tumor or cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRAS G12C mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G12S, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

### KRAS G12C Cancers

The methods described herein comprise treating a cancer with a KRAS G12C mutation in a patient. Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS.G12C tumors.” (Id., see also Canon et al., 2019, and Lanman et al., 2020).

Sotorasib was evaluated in a Phase 1 dose escalation and expansion trial with 129 patients having histologically confirmed, locally advanced or metastatic cancer with the KRAS G12C mutation identified by local molecular testing on tumor tissues, including 59 patients with non-small cell lung cancer, 42 patients with colorectal cancer, and 28 patients with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRAS G12C mutations occur with the alteration frequencies shown in the table below (Cerami et al., 2012; Gao et al., 2013). For example, the table shows that 11.6% of patients with non-small cell lung cancer have a cancer, wherein one or more cells express KRAS G12C mutant protein. Accordingly, sotorasib, which specifically and irreversibly bind to KRAS^{G12C} is useful for treatment of patients having a cancer, including, but not limited to the cancers listed in Table 1 below.

TABLE 1

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer. In some embodiments, the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer. In various embodiments, the cancer is non-small cell lung cancer, and in some embodiments, metastatic or locally advanced non-small cell lung cancer. In various embodiments, the cancer is colorectal cancer. In some embodiments, the cancer is pancreatic cancer. In some embodiments, the cancer is a solid tumor other than colorectal cancer or non-small cell lung cancer, for example, adenocarcinoma of the hepatic fexure with peritoneal carcinomatosis, adenocarcinoma for neuroendocrine features, solid tumor of metacarpal, ovarian solid tumor, pancreatic solid tumor, or small cell lung cancer.

### Prior Cancer Therapies of Patient Treated in Disclosed Methods

In some embodiments, the patient is a treatment-naïve patient—i.e., the patient has not received any prior therapy for the cancer having a KRAS G12C mutation being treated in the methods disclosed herein.

In some embodiments, the patient treated in the methods described herein has been previously treated with a different anti-cancer therapy, e.g., at least one—such as one, or two, three, or four—other systemic cancer therapy. In some embodiments, the patient had previously been treated with one other systemic cancer therapy, such that the therapy described herein is a second line therapy. In some embodiments, the patient had previously been treated with two other systemic cancer therapies, such that the therapy as provided herein is a third line therapy.

In some embodiments, the prior systemic cancer therapy is not a therapy with a KRAS^{G12C} inhibitor. In some embodiments, the prior systemic cancer therapy is a therapy with a KRAS^{G12C} inhibitor. In certain embodiments, the patient exhibits reduced sensitivity to a therapy with a KRAS^{G12C}. inhibitor. In some embodiments, the patient is resistant to a therapy with a KRAS^{G12C} inhibitor. In some embodiments, the KRAS^{G12C} inhibitor is sotorasib, adagrasib, GDC-6036, D-1553, JDQ443, LY3484356, BI1823911, JAB-21822, RMC-6291, or APG-1842. In certain embodiments, the KRAS^{G12C} inhibitor is sotorasib. In certain embodiments, the KRAS^{G12C} inhibitor is adagrasib. In some embodiments, the therapy is monotherapy. In one embodiment, the therapy with a KRAS^{G12C} inhibitor is sotorasib monotherapy. In another embodiment, the therapy with a KRAS^{G12C} inhibitor is monotherapy with adagrasib.

As used herein “sensitivity” refers to the way a cancer reacts to a drug, e.g., sotorasib and/or RMC-4630. In exemplary aspects, “sensitivity” means “responsive to treatment” and the concepts of “sensitivity” and “responsiveness” are positively associated in that a cancer or tumor that is responsive to a drug treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a cancer that is resistant to a drug treatment is neither sensitive nor responsive to that drug. In another example, a cancer that is resistant to a drug treatment was initially sensitive to the drug and is no longer sensitive upon acquiring resistance; thus, that drug is not or no longer an effective treatment for that tumor or cancer cell.

Other prior systemic cancer therapies include, but are not limited to, chemotherapies and immunotherapies. Specific contemplated prior systemic cancer therapies include, but are not limited to, anti-PD1 immunotherapy, anti-PD-L1 immunotherapy, and platinum-based chemotherapy. In some embodiments, the patient has not previously received an anti-PD1 or anti-PD-L1 immunotherapy. In some embodiments, the patient has previously received an anti-PD1 or anti-PD-L1 immunotherapy. Some examples of anti-PD1 immunotherapy and anti-PD-L1 immunotherapy include, but are not limited to, pembrolizumab, nivolumab, cemiplimab, tisielizumab, toripalimab, aspartalizumab, dostarlimab, retifanlimab, simtilimab, pidilizumab atezolizumab, avelumab, durvalumab, and zeluvalimab (AMG 404). Some examples of platinum-based chemotherapies include, but are not limited to, carboplatin, oxaliplatin, cisplatin, nedaplatin, satraplatin, lobaplatin, triplatin tetranitrate, picoplatin, ProLindac, and aroplatin. In some embodiments, the platinum-based chemotherapy is in combination with a second chemotherapeutic (e.g., a platinum-based combination chemotherapy), and such second chemotherapeutics can be, for example, paclitaxel, docetaxel, gemcitabine, or pemetrexed.

In some embodiments, the patient has an actionable oncogenic driver mutation in the epidermal growth factor receptor gene (EGFR), anaplastic lymphoma kinase gene (ALK), and/or ROS proto-oncogene 1 (ROS1) and previously received an EGFR, ALK, or ROS1 targeted therapy. In some embodiments, the patient progressed on an EGFR, ALK, or ROS1 targeted therapy. Targeted therapies for EGFR mutations include, but are not limited to, cetuximab, panitumumab, erlotinib, gefitinib, and afatinib. Targeted therapies for ALK mutations include, but are not limited to, crizotinib, entrectinib, lorlatinib, repotrectinib, brigatinib, alkotinib, alectinib, ensartinib, and ceritinib. Targeted therapies for ROS1 mutations include, but are not limited to, crizotinib, entrecetinib, ensartinib, alkotinib, brigatinib, taletrectinib, cabozantinib, repotrectinib, lorlatinib, and ceritinib.

In some embodiments, (1) (a) the patient previously received (i) anti-PD1 or anti-PD-L1 immunotherapy or (ii) platinum-based chemotherapy; or (b) the patient previously received (i) anti-PD1 or anti-PD-L1 therapy and (ii) platinum-based chemotherapy; and (2) the patient optionally has a mutation in EGFR, ALK, or ROS1 and previously received EGFR, ALK or ROS1 targeted therapy.

In various embodiments, the patient has non-small cell lung cancer, and (1) (a) the patient previously received (i) anti-PD1 or anti-PD-L1 immunotherapy or (ii) platinum-based chemotherapy; or (b) the patient previously received (i) anti-PD1 or anti-PD-L1 therapy and (ii) platinum-based chemotherapy; and (2) the patient optionally has a mutation in EGFR, ALK, or ROS1 and previously received an EGFR, ALK or ROS1 targeted therapy.

In various embodiments, that patient has colorectal cancer and (1) the patient received at least one prior systemic regime of therapy for advanced or metastatic colorectal cancer comprising fluoropyrimidine, oxaliplatin, or irinotecan-based therapy; and (2) the patient has an MSI-H cancer and received nivolumab or pembrolizumab, if the patient was clinically able to receive checkpoint inhibitors (e.g., PD1 or PD-L1 inhibitors). An MSI-H cancer refers to a cancer where the cells have high instability and stands for “microsatellite instability high.” Determination of MSI-H cancers can be assessed by the clinician using well known techniques, e.g., based upon the Bethesda panel-9, or as described, e.g., in U.S. Pat. Nos. 7,521,180; 7,662,595; 10,294,529; or 10,669,802.

In various embodiments, the patient did not receive a prior SHP2 inhibitor therapy. Examples of SHP2 inhibitors include, but are not limited to, RMC-4550, RMC-4630, SHP099, TNO155, JAB-3068, JAB-3312, RLY-1971, BBP-398, ERAS-601, PF-07284892, SH3809, ICP-189, and ET0038.

### Response to Sotorasib and RMC-4630 Therapy as Disclosed Herein

Response rates or results for patients administered the therapy (i.e., sotorasib and RMC-4630) in the methods disclosed herein can be measured in various ways, after the patient has been taking the therapy for a suitable length of time. In some embodiments, the therapy is administered in treatment cycles. In some embodiments, the treatment cycle is 21 days. In various embodiments, the patient undergoes 1, 2, 3, or more treatment cycles. In some embodiments, the patient undergoes at least 3 treatment cycles, at least 5 treatment cycles, at least 8 treatment cycles, at least 10 treatment cycles, or at least 15 treatment cycles.

In various embodiments, the patient is administered the therapy for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months. In various embodiments, the patient is administered the therapy for at least 1 month. In various embodiments, the patient is administered the therapy for at least 3 months. In various embodiments, the patient is administered the therapy for at least 6 months. In various embodiments, the patient is administered the therapy for at least 8 months.

The patient can respond to the therapy as measured by at least a stable disease (SD), as determined by Response Evaluation Criteria in Solid Tumors (RECIST) 1.1 protocol (Eisenhauer, et al., 2009). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Progression free survival (PFS) can be assessed as described in the RECIST 1.1 protocol. In various embodiments, the patient exhibits a PFS of at least 1 month. In various embodiments, the patient exhibits a PFS of at least 3 months. In some embodiments, the patient exhibits a PFS of at least 6 months.

Additional means for assessing response are described in detail in the examples below and can generally be applied to the methods disclosed herein.

### Embodiments

1. A method of treating cancer in a patient (i.e., a subject in need thereof), wherein the patient has a KRAS G12C mutation, the method comprising
administering to the patient a therapeutically effective combination of sotorasib and {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol (RMC-4630).

2. The method of embodiment 1, comprising administering 960 mg sotorasib daily.

3. The method of embodiment 1 or 2, comprising administering sotorasib orally once daily.

4. The method of any one of embodiments 1 to 3, comprising administering 50 to 200 mg RMC-4630.

5. The method of any one of embodiments 1 to 4, comprising administering 100 mg RMC-4630.

6. The method of any one of embodiments 1 to 4, comprising administering 140 mg RMC-4630.

7. The method of any one of embodiments 1 to 4, comprising administering 200 mg RMC-4630.

8. The method of any one of embodiments 1 to 4, comprising administering 50 mg RMC-4630.

9. The method of any one of embodiments 1 to 8, wherein RMC-4630 is administered orally on days 1 and 2 of each 7 days.

10 The method of any one of embodiments 1 to 8, wherein RMC-4630 is administered orally on days 1 and 4 of each 7 days.

11. The method of any one of embodiments 1 to 10, wherein RMC-4630 is administered to the patient in a fasted state.

12. The method of any one of embodiments 1 to 10, wherein RMC-4630 is administered to the patient after a 2 hour fast.

13. The method of any one of embodiments 1 to 12, wherein the patient fasts for 1 hour after the administration of RMC-4630.

14. The method of any one of embodiments 1 to 13, wherein sotorasib and RMC-4630 are administered for a treatment cycle, and the treatment cycle is 21 days.

15. The method of embodiment 14, wherein the patient undergoes at least 3 treatment cycles.

16. The method of embodiment 15, wherein the patient undergoes at least 8 treatment cycles.

17. The method of any one of embodiments 1 to 16, wherein the cancer is a solid tumor.

18. The method of any one of embodiments 1 to 17, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

19. The method of any one of embodiments 1 to 17, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

20. The method of any one of embodiments 1 to 17, wherein the cancer is non-small cell lung cancer.

21. The method of embodiment 20, wherein the non-small cell lung cancer is locally advanced or metastatic.

22. The method of any one of embodiments 1 to 17, wherein the cancer is colorectal cancer.

23. The method of any one of embodiments 1 to 17, wherein the cancer is a solid tumor other than colorectal cancer or non-small cell lung cancer.

24. The method of any one of embodiments 1 to 23, wherein the patient did not previously receive a prior line of anti-cancer therapy.

25. The method of any one of embodiments 1 to 23, wherein the patient received at least one prior line of anti-cancer therapy.

26. The method of any one of embodiments 1 to 25, wherein the patient did not previously receive an anti-PD1 or anti-PD-L1 immunotherapy.

27. The method of any one of embodiments 1 to 23, and 25, wherein the patient previously received an anti-PD1 or anti-PD-L1 immunotherapy.

28. The method of any one of embodiments1 to 23 and 25 to 27, wherein the patient previously received platinum-based chemotherapy.

29. The method of any one of embodiments 1 to 26, wherein the patient did not previously receive (i) anti-PD1 or anti-PDL1 immunotherapy or (ii) platinum-based chemotherapy.

30. The method of any one of embodiments 1 to 23, 27 and 28, wherein the patient previously received (i) anti-PD1 or anti-PD-L1 immunotherapy and (ii) platinum-based chemotherapy.

31. The method of any one of embodiments 1 to 23 and 25 to 30, wherein the patient (i) has a mutation in EGFR, ALK, or ROS1, and (ii) previously received an EGFR, ALK, or ROS1 targeted therapy.

32. The method of embodiment 31, wherein the patient progressed on the EGFR, ALK or ROS1 targeted therapy.

33. The method of embodiment any one of embodiments 1 to 17, wherein the cancer is non-small cell lung cancer, and
(1) (a) the patient previously received (i) anti-PD1 or anti-PD-L1 immunotherapy or (ii) platinum-based chemotherapy; or (b) the patient previously received (i) anti-PD1 or anti-PD-L1 therapy and (ii) platinum-based chemotherapy; and
(2) the patient optionally has a mutation in EGFR, ALK, or ROS1 and previously received an EGFR, ALK or ROS1 targeted therapy.

34. The method of any one of embodiments 1 to 17, wherein the cancer is colorectal cancer and
(1) the patient received at least one prior systemic regime of therapy for advanced or metastatic colorectal cancer comprising fluoropyrimidine, oxaliplatin, or irinotecan-based therapy; and
(2) the patient has a MSI-H cancer and received therapy with nivolumab or pembrolizumab, if the patient was clinically able to receive checkpoint inhibitors (e.g., PD1 or PDL1 inhibitors).

35. The method of any one of embodiments 1 to 34, wherein the patient did not receive a prior therapy with a KRAS^{G12C} inhibitor.

36. The method of any one of embodiments 1 to 23 and 25 to 34, wherein the patient received a prior therapy with a KRAS^{G12C} inhibitor.

37. The method of embodiment 36, wherein the patient progressed on the prior therapy with the KRAS^{G12C} inhibitor.

38. The method of any one of embodiments 35 to 37, wherein the KRAS^{G12C} inhibitor is sotorasib or adagrasib.

39. The method of any one of embodiments 1 to 38, wherein the patient has an Eastern Cooperative Oncology Group (ECOG) performance status of less than or equal to 1.

40. The method of any one of embodiments 1 to 39, wherein the patient does not have a primary brain tumor.

41. The method of any one of embodiments 1 to 40, wherein the patient does not have spinal cord compression or untreated or symptomatic brain metastases or leptomeningeal disease from nonbrain tumors.

42. The method of any one of embodiments 1 to 41, wherein
(i) the patient
had brain metastases resected, or
received whole brain radiation therapy ending at least 4 weeks prior to start of first treatment cycle, or
received stereotactic radiosurgery ending at least 2 weeks prior to first treatment cycle, and
(ii) the patient
exhibits residual neurological symptoms of grade 2 or less, and
receives a stable dose of dexamethasone, if applicable; and
has an magnetic resonance imaging (MRI) performed within 30 days prior to first treatment cycle shows no new or enlarging lesions appearing.

43. The method of any one of embodiments 1 to 42, wherein the patient does not have either 160 mmHg or greater systolic blood pressure or 100 mmHg or greater diastolic blood pressure.

44. The method of any one of embodiments 1 to 43, wherein the patient is in further need of treatment with a CYP3A4 substrate.

45. The method of embodiment 44, wherein the patient is not administered a CYP3A4 substrate during any treatment cycle or within 14 days or 5 half-lives of the CYP3A4 substrate or its major active metabolite, whichever is longer, before starting administration of sotorasib and RMC-4630.

46. The method of embodiment 44 or 45, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, imatinib, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus (fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, or ziprasidone.

47. The method of embodiment 44 or 45, wherein the CYP3A4 substrate is a CYP3A4 substrate with a narrow therapeutic index.

48. The method of embodiment 44, 45 or 47, wherein the CYP3A4 substrate is alfentanil, cyclosporine, dihydroergotamine, ergotamine, everolimus, fentanyl, primozide, quinidine, tacrolimus, or sirolimus.

49. The method of any one of embodiments 1 to 48, wherein the patient is in further need of treatment with a P-glycoprotein (P-gp) substrate.

50. The method of embodiment 49, wherein the patient is not administered a P-gp substrate during any treatment cycle or within 14 days or 5 half-lives of the P-gp substrate or its major active metabolite, whichever is longer, before starting administration of sotorasib and RMC-4630.

51. The method of embodiment 49 or 50, wherein the P-gp substrate is etexilate, digoxin, fexofenadine, everolimus, cyclosporine, sirolimus, tacrolimus, or vincristine.

52. The method of embodiment 49 or 50, wherein the P-gp substrate is a P-gp substrate with a narrow therapeutic index.

53. The method of embodiment 49, 50, or 52, wherein the P-gp substrate is digoxin, everolimus, cyclosporine, tacrolimus, sirolimus, or vincristine.

54. The method of any one of embodiments 1 to 53, wherein the patient is in further need of treatment with a strong CYP3A4 inducer.

55. The method of embodiment 54, wherein the patient is not administered a strong CYP3A4 inducer during any treatment cycle or within 14 days or 5 half-lives of the strong CYP3A4 inducer, whichever is longer, before starting administration of sotorasib and RMC-4630.

56. The method of embodiment 54 or 55, wherein the strong CYP3A4 inducer is rifampin, mitotane, avasimibe, rifapentine, apalutamide, phenytoin, carbamazepine, enzalutamide, ivosidenib, St. John's wort extract, or lumacaftor.

57. The method of any one of embodiments 1 to 56, wherein the patient has not received prior SHP2 inhibitor therapy.

58. The method of embodiment 57, wherein the SHP2 inhibitor is RMC-4550, RMC-4630, SHP099, TNO155, JAB-3068, JAB-3312, RLY-1971, BBP-398, ERAS-601, PF-07284892, SH3809, ICP-189, or ET0038.

59. The method of any one of embodiments 1 to 58, wherein the patient is in further need of treatment with a proton pump inhibitor (PPI) or a H2 receptor antagonist (H2RA).

60. The method of embodiment 59, wherein the patient is not administered a PPI or a H2RA during any treatment cycle or within three days of starting administration of sotorasib and RMC-4630.

61. The method of embodiment 59 or 60, wherein the PPI is lansoprazole, omeprazole, pantoprazole, raveprazole, or esomeprazole.

62. The method of embodiment 59, 60, or 61, wherein the H2RA is cimetidine, famotidine, nizatidine, or ranitidine.

63. The method of any one of embodiments 1 to 62, wherein the patient exhibits at least a stable disease (SD), as measured by RECIST 1.1 protocol, after at least 3, 6, or 8 months.

64. The method of any one of embodiments 1 to 63, wherein the patient exhibits at least a partial response (PR), as measured by RECIST 1.1 protocol, after at least 3, 6, or 8 months.

65. The method of any one of embodiments 1 to 64, wherein the patient exhibits a progression free survival (PFS) of at least 3 months.

### Examples

This is a phase 1b multicenter, open label study evaluating the safety, tolerability, pharmacokinetics, and efficacy of sotorasib in combination with RMC-4630 in subjects with KRAS G12C mutant advanced solid tumors.

### Part 1

Part 1 of the study will be aimed at assessing the safety and tolerability of the selected starting dose of sotorasib and RMC-4630 as combination therapy. The starting dose for sotorasib and RMC-4630 is based on the review of the safety and PK data from the phase 1/2 Study 20170543 (NCT03600883) exploring sotorasib as a monotherapy and the review of the safety and PK data from a phase 1 monotherapy study for RMC-4630 (NCT03634982)

The recommended phase 2 dose (RP2D) for RMC-4630 monotherapy is 200 mg D1D2 (i.e., administered on Day 1 and Day 2) of each 7 days. Note that D1D4 indicates administration of Day 1 and Day 4 of each 7 days. The planned dose levels for RMC-4630 with a fixed dose of sotorasib (960 mg orally once daily) are provided below:
Dose Level 1:100 mg (D1D4 and/or D1D2 of each 7 days)
Dose Level −1: up to 50% reduction from Level 1 (D1D2 of each 7 days)
Dose Level 2:140 mg (D1D2 of each 7 days)
Dose Level 3:200 mg (D1D2 of each 7 days)

Dose exploration will begin with treating 3-4 subjects at Dose Level 1. The study dose-limiting toxicity (DLT) period is 21 days. Once all subjects enrolled at a certain dose level and schedule are DLT evaluable, a Dose Level Review Team (DLRT) meeting will be convened. For any dose level (including Dose Level 1), and depending on observed safety data, the following may occur: 1) dose escalation to the next highest dose level or 2) additional enrollment to the current dose level or 3) dose de-escalation to the next lowest dose level. Rules for dose-escalation/de-escalation are derived using a modified Toxicity Probability Interval-2 (mTPI-2) model (Guo et al, 2017) with a target toxicity probability of 0.30 (Table 2)

TABLE 2

| No. of | Number of subjects with a | |
|---|---|---|
| Evaluable^{a} | DLT observed at current | Guideline for Dose Escalation/ |
| Subjects | dose level and schedule | De-escalation |
| 3 | 0 | Escalate dose level^{b} |
| 3 | 1 | Stay at current dose level |
| 3 | ≥2 | De-escalate or stop enrollment at |
| | | current dose level^{b} |
| 4 | 0 | Escalate dose level^{b} |
| 4 | 1 | Stay at current dose level |
| 4 | ≥2 | De-escalate or stop enrollment at |
| | | current dose level^{b} |
| 5 | ≤1 | Escalate dose level^{b} |
| 5 | ≥2 | De-escalate or stop enrollment at |
| | | current dose level^{b} |
| 6 | ≤1 | Escalate dose level^{b} |
| 6 | ≥2 | De-escalate or stop enrollment at |
| | | current dose level^{b} |
| 7 or 8^{c} | ≤1 | Escalate dose level^{b} |
| 7 or 8^{c} | 2 | Stay at current dose level |
| 7 or 8^{c} | ≥3 | De-escalate or stop enrollment at |
| | | current dose level^{b} |
| 9^{c} | ≤2 | Escalate dose level^{b} |
| 9^{c} | ≥3 | De-escalate or stop enrollment at |
| | | current dose level^{b} |
| 10-12^{c} | ≤2 | Escalate dose level^{b} |
| 10-12^{c} | 3 | Stay at current dose level |
| 10-12^{c} | ≥4 | De-escalate or stop enrollment at |
| | | current dose level^{b} |
| 13-15^{c} | ≤3 | Escalate dose level^{b} |
| 13-15^{c} | 4 | Stay at current dose level |
| 13-15^{c} | ≥5 | De-escalate or stop enrollment at |
| | | current dose level^{b} |
| 16^{c} | ≤3 | Escalate dose level^{b} |
| 16^{c} | 4 or 5 | Stay at current dose level |
| 16^{c} | ≥6 | De-escalate or stop enrollment at |
| | | current dose level^{b} |
| 17-18^{c} | ≤4 | Escalate dose level^{b} |
| 17-18^{c} | 5 | Stay at current dose level |
| 17-18^{c} | ≥6 | De-escalate or stop enrollment at |
| | | current dose level^{b} |
| 19-20^{c} | ≤4 | Escalate dose level^{b} |
| 19-20^{c} | 5 or 6 | Stay at current dose level |
| 19-20^{c} | ≥7 | De-escalate or stop enrollment at |
| | | current dose level^{b} |

DLT = dose-limiting toxicity
^{a}A subject is evaluable if subject experiences a DLT or completes 21 days on treatment and receives at least 80% of planned cumulative dose of sotorasib and at least 4 full doses of 6 planned doses of RMC-4630 during the DLT period. A subject without a DLT will not be DLT evaluable if the date of decision to end both investigational products is before the completion of the DLT window.
^{b}De-escalation guideline applies only when enrollment is allowed to a lower dose level. Re-escalation may be allowed, as appropriate, only in the following instances: 2 of 5 or 2 of 6 evaluable subjects experience a DLT, 3 of 8 or 3 of 9 subjects experience a DLT, 4 of 10-12 subjects experience a DLT, 5 of 13-15 subjects experience a DLT, 6 of 16-18 subjects experience a DLT, or 7 of 19-20 subjects experience a DLT.
^{c}As appropriate to better understand safety profile for a dose level, the number of evaluable subjects may be expanded up to 20.

In addition to the planned dose levels, further degree of dose modification (e.g., intermediate doses) and/or schedule of administration (e.g., alternate dosing) will be decided based on analysis of emerging safety and PK data. The maximum tolerated dose (MTD) will be estimated using isotonic regression (Ji et al, 2010) and the MTD will be the dose level with the estimated DLT rate closest to 0.30. In order to consider a certain Dose Level as the MTD or recommended safe combination dose for Part 2, at least 6 DLT evaluable subjects must be enrolled at that dose level. No more than 20 DLT evaluable subjects will be enrolled at any specific dose level and schedule in Part 1. A DLRM will convene to review data after a total of 10 to 15 subjects have enrolled at a single dose level in Part 1 and become DLT evaluable.

Part 1 will end once any of the following events occur:

The highest dose level is determined to be safe and tolerable (minimum of 6 evaluable subjects overall).

A dose level is determined to be safe and tolerable (minimum of 6 evaluable subjects) and the next higher dose level is determined to be unsafe and intolerable.

All planned dose levels are determined to be unsafe and intolerable.

On the basis of a review of real-time safety data and available preliminary PK data, dose escalation may be halted or modified by the sponsor as deemed appropriate.

### Part 2

Upon completing the dose exploration part of the study and depending on data obtained, enrollment will commence in the dose expansion phase (Part 2) to confirm the safety and tolerability of the selected dose and to further evaluate anti-tumor activity. Part 2 will consist of two groups.

Group 2b will consist of up to 20 subjects with advanced CRC with KRAS G12C mutation.

Group 2c will consist of up to 20 subjects with advanced solid tumors (other than NSCLC or CRC) with KRAS G12C mutation.

For both Part 1 and Part 2, administration of sotorasib and RMC-4630 may continue until evidence of disease progression, intolerance to study medication, withdrawal of consent, or end of study.

### Endpoints:

| | |
|---|---|
| Objectives | Endpoints |

Primary

| | |
|---|---|
| To evaluate the safety and tolerability of | Dose-limiting toxicities (DLTs), treatment- |
| sotorasib in combination with RMC-4630 | emergent adverse events, treatment- |
| in adult subjects with KRAS G12C mutant | related adverse events, and changes in |
| advanced solid tumors | vital signs, electrocardiograms, and |
| | clinical laboratory tests |

Secondary

| | |
|---|---|
| To characterize the pharmacokinetics | PK parameters of sotorasib and RMC- |
| (PK) of sotorasib and RMC-4630 following | 4630 including, but not limited to |
| administration as combination therapy in | maximum plasma concentration (Cₘₐₓ), |
| adult subjects with KRAS G12C mutant | time to maximum concentration (tₘₐₓ), and |
| advanced solid tumors | area under the concentration-time curve |
| | (AUC) |
| To evaluate anti-tumor activity of | Objective response rate, duration of |
| sotorasib in combination with RMC-4630 | response, time to response, overall |
| in adult subjects with KRAS G12C mutant | survival (OS), progression-free survival, |
| advanced solid tumors | disease control rate, and duration of |
| | stable disease measured by computed |
| | tomography or magnetic resonance |
| | imaging and assessed per RECIST v1.1 |

Exploratory

| | |
|---|---|
| To explore PK relationships for safety | Sotorasib and RMC-4630 exposure/safety |
| and/or efficacy endpoints | and exposure/efficacy relationships |
| To investigate potential biomarkers by | Quantification of biomarker expression at |
| biochemical and/or genetic analysis of | protein, RNA, and DNA levels, as |
| blood and/or tumor tissue samples | appropriate in blood and/or tumor |
| | samples |

### Inclusion Criteria:

Subject has provided informed consent prior to initiation of any study specific activities/procedures.

Male and female subjects age ≥18 years old.

Pathologically documented, locally-advanced, or metastatic malignancy with KRAS G12C mutation identified through molecular testing.

For NSCLC, subjects must have received anti-PD-1 or anti-PD-L1 therapy (unless contraindicated) AND/OR platinum-based combination chemotherapy AND targeted therapy (if actionable oncogenic driver mutations were identified [e.g., EGFR, ALK, and ROS1]), or if subject refused standard therapy. Prior neoadjuvant/adjuvant chemotherapy will be considered for eligibility only if the subject progressed on or within 6 months of completing the therapy. KRAS G12C mutation must be identified by an approved diagnostic device for detection of KRAS G12C in NSCLC. In the United States, this test must be performed in a Clinical Laboratory Improvement amendments (CLIA) certified laboratory.

For CRC, all subjects must have received at least one prior systemic regimens of therapy for advanced or metastatic CRC including fluoropyrimidine, oxaliplatin, and irinotecan-based regimens. In addition, subjects with tumors that are MSI-H must have received treatment with either nivolumab or pembrolizumab if they were clinically able to receive checkpoint inhibitors and if one of these agents is approved for that indication in the region or country, or if the subject refused standard therapy.

For advanced solid tumor types other than NSCLC and CRC, subjects must have received at least one prior systemic therapy and be intolerant or ineligible for available therapies known to provide clinical benefit (Part 1 only).

Subjects who have received prior KRAS G12C targeted therapy must have progressed (Part 1 Only)

Subjects must be willing to undergo pretreatment tumor biopsy if clinically feasible. If a tumor biopsy prior to treatment is not medically feasible, or if the sample has insufficient tissue for testing, subjects must be willing to provide archived tumor tissue samples (formalin fixed paraffin embedded [FFPE] sample collected within the past 3 years. Subjects who do not have archived tissue available can be allowed to enroll without undergoing tumor biopsy upon agreement with investigator and the Medical Monitor if a tumor biopsy is not feasible. Subjects must also be willing to undergo a tumor biopsy, if medically feasible, on a dosing day between week 2 to week 5 after starting treatment.

Measurable disease per RECIST v1.1 criteria.

Eastern Cooperative Oncology Group (ECOG) Performance Status of ≤1.

Life expectancy of >3 months, in the opinion of the investigator.

Ability to take oral medications and willing to record daily adherence to investigational products.

Corrected QT interval (QTc)≤470 msec for females and ≤450 msec for males (based on average of screening triplicates).

Adequate hematological laboratory assessments, as follows:
Absolute neutrophil count (ANC)≥1.5×10⁹/L
Platelet count ≥100×10⁹/L
Hemoglobin ≥9 g/dL

Adequate renal laboratory assessments, e.g., Estimated glomerular filtration rate based on Modification of Diet in Renal Disease calculation ≥60 ml/min/1.73 m².

Adequate hepatic laboratory assessments, as follows:
AST <2.5× upper limit of normal (ULN) (if liver metastases are present, ≤5×ULN)
ALT <2.5×ULN (if liver metastases are present, ≤5×ULN)
Total bilirubin <1.5×ULN (<2.0×ULN for subjects with documented Gilbert's syndrome or <3.0×ULN for subjects for whom the indirect bilirubin level suggests an extrahepatic source of elevation)

Adequate coagulation laboratory assessments, as follows:
Prothrombin time and (activated) partial thromboplastin time <1.5×ULN, OR International normalized ratio <1.5 or within target range if on prophylactic anticoagulation.

Cardiac ejection fraction ≥50%, with no evidence of pericardial effusion as determined by an echocardiogram or multigated acquisition scan.

### Exclusion Criteria:

### Primary Brain Tumor.

Spinal cord compression or untreated or symptomatic brain metastases or leptomeningeal disease from non-brain tumors. Subjects who have had brain metastases resected or have received whole brain radiation therapy ending at least 4 weeks (or stereotactic radiosurgery ending at least 2 weeks) prior to study day 1 are eligible if they meet all of the following criteria: a) residual neurological symptoms grade≤2; b) on stable doses of dexamethasone, if applicable; and c) follow-up magnetic resonance imaging (MRI) performed within 30 days shows no new or enlarging lesions appearing.

History or presence of hematological malignancies unless curatively treated with no evidence of disease ≥2 years.

History of other malignancy within the past 2 years, with the following exceptions:
Malignancy treated with curative intent and with no known active disease present for ≥2 years before enrollment and felt to be at low risk for recurrence by the treating physician.
Adequately treated non-melanoma skin cancer or lentigo maligna without evidence of disease.
Adequately treated cervical carcinoma in situ without evidence of disease.
Adequately treated breast ductal carcinoma in situ without evidence of disease.
Prostatic intraepithelial neoplasia without evidence of prostate cancer.
Adequately treated urothelial papillary non-invasive carcinoma or carcinoma in situ.

Myocardial infarction within 6 months of study day 1, symptomatic congestive heart failure (New York Heart Association>class II), unstable angina, or clinically significant findings on ECG.

Gastrointestinal tract disease causing the inability to take oral medication, malabsorption syndrome, requirement for intravenous (IV) alimentation, uncontrolled inflammatory GI disease (e.g., Crohn's disease, ulcerative colitis).

Exclusion of hepatitis infection based on the following results and/or criteria:
Positive Hepatitis B Surface Antigen (HepBsAg) (indicative of chronic hepatitis B or recent acute hepatitis B).
Negative HepBsAg with a positive for hepatitis B core antibody (Hepatitis B core antibody testing is not required for screening, however if this is done and is positive, then hepatitis B surface antibody [Anti-HBs] testing is necessary. Undetectable anti-HBs in this setting would suggest unclear and possible infection, and needs exclusion).
Positive Hepatitis C virus antibody: Hepatitis C virus RNA by polymerase chain reaction is necessary. Detectable Hepatitis C virus RNA suggests chronic hepatitis C.

Positive test for human immunodeficiency virus (HIV).

Medically uncontrolled hypertension (≥160 mmHg systolic blood pressure or ≥100 mmHg diastolic blood pressure).

History or current evidence of retinal pigment epithelial detachment (RPED), central serous retinopathy (CSR), retinal vein occlusion (RVO), or predisposing factors to RPED or RVO (e.g., uncontrolled glaucoma or ocular hypertension, uncontrolled diabetes mellitus, hyperviscosity, or hypercoagulability syndromes).

Visible retinal pathology as assessed on ophthalmic examination that is considered a significant risk factor for RVO or RPED by ophthalmologist.

Presence of grade ≥2 proteinuria.

Active, clinically significant interstitial lung disease or pneumonitis.

Active autoimmune disease requiring systemic treatment within the past 2 years (ie, with the use of disease modifying agents, non-physiologic doses of corticosteroids or immunosuppressive drugs); including current autoimmune sequelae or previous grade ≥2 autoimmune sequelae from checkpoint inhibitors or other immunomodulatory treatments that require systemic therapy. Exception: Subjects with autoimmune endocrine disorders on hormonal supplementation may be enrolled even if>grade 2 when it first occurred, if approved by the Medical Monitor.

History of severe allergic reactions to any of the study intervention components.

Presence of grade≥2 systemic edema or ascites.

Prior tumor therapy (chemotherapy, antibody therapy, molecular targeted therapy, retinoid therapy, hormonal therapy [except for subjects with breast cancer], or investigational agent) within 28 days of study day 1; concurrent use of hormone deprivation therapy for hormone-refractory prostate cancer or breast cancer is permitted. Exception: subjects who receive prior targeted small molecule inhibitors, sotorasib monotherapy or conventional chemotherapy within 14 days of study day 1.

Therapeutic or palliative radiation therapy within 2 weeks of study day 1. Subjects must have recovered from all radiotherapy related toxicity.

Use of known cytochrome P450 (CYP) 3A4 and P-glycoprotein (P-gp) sensitive substrates (with a narrow therapeutic window), within 14 days or 5 half-lives of the drug or its major active metabolite, whichever is longer, prior to study day 1 that was not reviewed and approved by the principal investigator and the Medical Monitor.

Use of strong inducers of CYP3A4 (including herbal supplements such as St. John's wort) within 14 days or 5 half-lives (whichever is longer) prior to study day 1 that was not reviewed and approved by the principal investigator and the Medical Monitor.

Active infection requiring intravenous (IV) antibiotics within 1 week of study enrollment (day 1).

Unresolved toxicities from prior anti-tumor therapy, defined as not having resolved to CTCAE version 5.0 grade 0 or 1, or to levels dictated in the eligibility criteria with the exception of alopecia (grade 2 or 3 toxicities from prior anti-tumor therapy that are considered irreversible [defined as having been present and stable for >6 months), such as ifosfamide-related proteinuria or neuropathy, may be allowed if they are not otherwise described in the exclusion criteria AND there is agreement to allow by both the investigator and sponsor).

Subject unable to receive both iodinated contrast for computed tomography (CT) scans and gadolinium contrast for MRI scans.

Any dose reduction, ≥grade 3 treatment-related adverse event, or treatment discontinuation for any adverse event related to the previous treatment with KRAS^{G12C} inhibitor (only for subjects enrolled in Part 1).

Prior SHP2 inhibitor therapy (both Part 1 and Part 2) and prior KRAS^{G12C} inhibitor therapy (only for subjects enrolled in Part 2).

Major surgical procedures≤28 days or non-study-related minor procedures ≤7 days prior to cycle 1 day 1. In all cases, the subjects must be sufficiently recovered and stable before treatment administration.

Use of proton pump inhibitors (PPIs) within 3 days or H2-receptor antagonists (H2RAs) within 1 day prior to study intervention and requirement for treatment with a PPI or H2-receptor antagonist during the dose escalation portion of the study.

Currently receiving treatment in another investigational device or drug study, or less than 28 days since last intervention on another investigational device or drug study(ies). Other investigational procedures while participating in this study are excluded.

Subject has known sensitivity to any of the products or components to be administered during dosing.

Subject likely to not be available to complete all protocol-required study visits or procedures, and/or to comply with all required study procedures (e.g., Clinical Outcome Assessments) to the best of the subject and investigator's knowledge.

History or evidence of any other clinically significant disorder, condition or disease (with the exception of those outlined above) that, in the opinion of the investigator or physician, if consulted, would pose a risk to subject safety or interfere with the study evaluation, procedures or completion.

Female subjects of childbearing potential with a positive pregnancy test assessed at Screening or day 1 by a serum pregnancy test and/or urine pregnancy test.

Female subject is pregnant or lactating/breastfeeding or planning to become pregnant or breastfeed during treatment and for an additional 7 days after the last dose of sotorasib.

Female subjects of childbearing potential unwilling to use 1 highly effective method of contraception during treatment and for an additional 7 days after the last dose of sotorasib.

Female subjects pregnant or lactating/breastfeeding or planning to become pregnant or breastfeed during treatment and for an additional 2 months after the last dose of RMC-4630.

Female subjects of childbearing potential unwilling to use 1 highly effective method of contraception during treatment and for an additional 2 months after the last dose of RMC-4630.

Male subjects with a pregnant partner who are unwilling to practice abstinence or use a condom during treatment and for an additional 7 days after the last dose of sotorasib.

Male subjects who are unwilling to abstain from donating sperm during treatment and for an additional 7 days after the last dose of sotorasib.

Male subjects with a female partner of childbearing potential who are unwilling to practice sexual abstinence (refrain from heterosexual intercourse) or use contraception during treatment and for an additional 3 months after the last dose of RMC-4630.

Male subjects who are unwilling to practice abstinence or use a condom during treatment and for an additional 3 months after the last dose of RMC-4630.

Male subjects who are unwilling to abstain from donating sperm during treatment and for an additional 3 months after the last dose of RMC-4630.

Dosing instructions for sotorasib: Sotorasib will be administered orally once daily (QD) for a treatment cycle of 21 days. Subject should take the sotorasib dose (all tablets at the same time) at approximately the same time every day. The sotorasib dose should not be taken more than 2 hours earlier than the target time based on the previous day's dose. The sotorasib dose should also not be taken more than 6 hours after the target time based on the previous day's dose. The next dose should be taken as prescribed. If vomiting occurs after taking sotorasib, the subject should not take an additional dose. The next dose should be taken as prescribed. Administration to patients who have difficulty swallowing solids disperse tablets in 120 mL (4 ounces) of non-carbonated, room-temperature water without crushing. No other liquids should be used. Stir until tablets are dispersed into small pieces (the tablets will not completely dissolve) and drink immediately or within 2 hours. The appearance of the mixture may range from pale yellow to bright yellow. Swallow the tablet dispersion. Do not chew pieces of the tablet. Rinse the container with an additional 120 mL (4 ounces) of water and drink. If the mixture is not consumed immediately, stir the mixture again to ensure that tablets are dispersed.

Dosing instructions for RMC-4630: RMC-4630 will be administered orally twice a week on days 1 and 2 or days 1 and 4, taken with water after a 2 hour fast; no food or drink other than water is allowed for 1 hour after administration. Antacids are permitted but should not be consumed within 3 to 4 hours before or after RMC-4630 administration. If a patient misses a dose, it may be taken at the time the omission was discovered provided that it is within 24 hours of the original administration time. Otherwise, the dose should not be administered and should be recorded as an omission. The treatment cycle is the same as that of sotorasib. A dose of RMC-4630 can be replaced in the event of vomiting if the vomiting occurs within 15 minutes of the dosing, all tablets administered have been accounted for (e.g., 4 tablets must be collected if 4 tablets were administered) and are intact by visual inspection (not broken, partially dissolved, chewed, or crushed), and the investigator deems it safe to replace the dose. Please note that the sotorasib dose should not be similarly replaced.

On days that sotorasib and RMC-4630 are scheduled to be administered, both drugs should be taken together with 1 cup of water. No food or liquids except water are allowed 2 hours before to 1 hour after dosing with RMC-4630.

Excluded Treatments, Medical Devices, and/or Procedures During Study Period:

Anti-tumor therapy such as chemotherapy, antibody therapy, molecular targeted therapy, retinoid therapy, and hormonal therapy (except for subjects with breast and prostate cancer receiving it as adjuvant therapy).

Known CYP3A4 and P-gp sensitive substrates with a narrow therapeutic window unless approved by medical monitor.

Strong CYP3A4 inducers including herbal supplements such as St John's wort) unless approved by the medical monitor.

Medications known to prolong QTc interval (e.g., amiodarone, azithromycin, cilostazol, disopyramide, dronedarone, escitalopram, haloperidol, methadone, oxaliplatin, procainamide, sevoflurane, vandetanib, anagrelide, chloroquine, ciprofloxacin, dofetilide, droperidol, flecainide, ibutilide, moxifloxacin, pentamidine, propofol, sotalol, arsenic trioxide, chloropromazine, citalopram, donepezil, erythromycin, fluconazole, levofloxacin, ondansetron, pimozide, quinidine and/or thioridazine).

Proton pump inhibitors and H2-receptor antagonists unless approved by medical monitor.

Other investigational agents.

Therapeutic or palliative radiation therapy to non-target lesion(s) may be allowed for symptom control provided there is discussion and agreement between investigator and medical monitor prior to radiation therapy. Study drugs must be held during radiation therapy.

Subjects must not schedule any major elective surgeries during the treatment period, and for at least 28 days after the last administration of sotorasib. Minor elective surgery may be allowed after discussion with the medical monitor. If a subject undergoes any unexpected surgery during the course of the study, all study drugs must be discontinued, and the investigator or designee should notify the sponsor as soon as possible. A subject may be allowed to resume sotorasib only if both the investigator and medical monitor agree to restart study therapy.

### Dose-Limiting Toxicities

The DLT window (i.e., DLT-evaluable period) will be the first 21 days of sotorasib and RMC-4630 treatment (starting cycle 1, day 1). The grading of adverse events will be based on the guidelines provided in the CTCAE version 5.0. A subject will be DLT evaluable if the subject has completed the DLT window as described above and received at least 80% of the planned cumulative dose of sotorasib and at least 4 full doses of 6 planned doses of RMC-4630 or experienced a DLT any time during the DLT window. A subject without a DLT will not be DLT evaluable if the date of decision to end both investigational products is before the completion of the DLT window. Dose-limiting toxicity is defined as any adverse event meeting the criteria listed below occurring during the first treatment cycle and attributable to sotorasib and/or RMC-4630.

An adverse event that results in permanent discontinuation of any investigation product.

An adverse event that results in a delay in the start of cycle 2 by >2 weeks.

Febrile neutropenia.

Grade 4 neutropenia of any duration.

Grade 3 neutropenia lasting >7 days.

Grade 3 thrombocytopenia with grade ≥2 bleeding.

Grade 4 thrombocytopenia.

Grade 4 anemia.

Grade ≥4 vomiting and diarrhea.

Grade ≥3 vomiting or diarrhea lasting more than 3 days despite optimal medical support.

Grade ≥3 nausea lasting 3 days or more despite optimal medical support.

Grade ≥3 rash lasting 3 days or more despite optimal medical support.

Grade 3 AST or ALT elevation lasting >5 days or grade 4 AST or ALT elevation of any duration.

Grade ≥3 bilirubin elevation.

Grade ≥3 QTc prolongation.

Retinal vein occlusion.

Any other grade ≥3 adverse event with the following exceptions:
fatigue
asymptomatic grade 3 electrolyte abnormalities that last <72 hours, are not clinically complicated, and resolve spontaneously or respond to medical interventions
grade 3 amylase or lipase that is not associated with symptoms or clinical manifestations of pancreatitis
other select lab abnormalities that do not appear to be clinically relevant or harmful to the patient and/or can be corrected with replacement or modifications (e.g., grade 3 lymphopenia, grade 3 hypoalbuminemia).

Any subject meeting the criteria for Hy's Law case (i.e., severe drug-induced liver injury [DILI]) will be considered a DLT. A Hy's Law case is defined as: AST or ALT values of ≥3×ULN AND with serum total bilirubin level of >2×ULN without signs of cholestasis and with no other clear alternative reason to explain the observed liver-related laboratory abnormalities.

If a subject experiences a DLT during the DLT evaluation period, or any treatment related toxicity meeting the DLT definition after day 21 sotorasib and/or RMC-4630 treatment should be modified as discussed below.

### Dose Adjustments, Delays, Rules for Withholding or Restarting, Permanent Discontinuation

Dose reduction levels of sotorasib for toxicity management of individual subjects are provided in Table 3. Up to two dose reductions are allowed. Dose reductions below 240 mg are not allowed.

TABLE 3
Sotorasib Dose Levels (mg QD)

| Starting Dose | Dose -1 | Dose -2 |
|---|---|---|
| 960 | 480 | 240 |

Sotorasib will be discontinued or the dosage reduced, in the event of a toxicity that, in the opinion of the investigator, warrants the discontinuation, or dose reduction as indicated in Table 4. Subjects who experience an adverse event requiring dose reductions below 240 mg should be permanently discontinued from sotorasib treatment.

TABLE 4

| | Recommended Action | |
|---|---|---|
| Toxicity | Hold Until: | Restart Dose: |
| Grade ≥3 nausea, vomiting, or | Recovery to grade 1 or | Resume dosing at 1 dose |
| diarrhea lasting longer than | less or to baseline grade | lower^{a} |
| 3 days despite optimal medical | | |
| support | | |
| Any other drug-related | Recovery to grade 1 or | Resume dosing at 1 dose |
| toxicity ≥grade 3^{b} | less or to baseline grade | lower^{a} |
| Suspected interstitial | ILD/pneumonitis confirmed | If confirmed, permanently |
| lung disease (ILD)/ | or excluded | discontinue sotorasib |
| pneumonitis of any grade | | If excluded, restart at same dose |
| | | if no other sotorasib dose |
| | | modification guidelines are |
| | | applicable |

^{a}Subjects may be resumed at a dose lower than the recommended restarting dose after discussion with the Medical Monitor. Dose re escalation after a dose reduction (except as a result of a DLT) may be allowable only after discussion with Medical Monitor.
^{b}For suspected hepatotoxicity, see below.

If the initiation of a new cycle to be is delayed, the ongoing cycle will be continued until the following cycle is started. The start of the following cycle begins with reinitiating of study treatment. Once the following cycle is started, the 21-day cycle duration should be restored and maintained. However, if a dose within a cycle is held or missed, the missed dose will not be made up, and day 1 of subsequent cycles should not be adjusted.

If the adverse event is suspected to be due to RMC-4630, the following dose modification criteria can be used as a guide to either continue, interrupt, or discontinue treatment permanently. Any adverse event/serious adverse event that does not resolve within 21 days from onset may result in permanent discontinuation of treatment with RMC-4630 upon investigator's discretion. Dose reduction levels of RMC-4630 for toxicity management of individual subjects will be similar to the dose levels being explored under Part 1.

Grade 1 and 2: Continue study intervention at current dose level and monitor closely. For grade 2 adverse events, study drug may be interrupted. Provide supportive care according to institutional guidelines.

Grade 3 (meets DLT criteria): Interrupt or discontinue study treatment as deemed medically appropriate and monitor closely. Provide supportive care according to institutional guidelines. When toxicity resolves to grade 1 or baseline, treatment can be restarted at a lower dose level after the DLT evaluation period is complete and the patient is deriving clinical benefit.

Grade 3 (does not meet DLT criteria): Interrupt or discontinue study treatment as deemed medically appropriate and monitor closely. Provide supportive care according to institutional standards. For some adverse events such as anemia, dose interruption or discontinuation may not be required depending on baseline hemoglobin and transfusion history. When toxicity resolves to grade 1 or baseline, restart study treatment at current dose level if clinically appropriate (such as in the case of uncomplicated thrombocytopenia without bleeding and anemia). If the grade 3 toxicity recurs, either permanently discontinue study treatment or, if the subject is clinically benefiting, consider continuation of study treatment at a lower dose.

Grade 4: Discontinue study treatment permanently (for most adverse events/serious adverse events unless investigator believes it to be an isolated incident and clinically insignificant) and monitor closely. Provide supportive care according to institutional standards. Only if the subject is clinically benefitting, treatment can be continued at a lower dose.

### Intrasubject Dose Escalation:

Certain subjects who enroll in the study and receive at least 80 percent of the planned dose for sotorasib and at least 8 out of 12 doses for RMC-4630 over a period of at least 2 cycles will be allowed to receive a higher dose of RMC-4630 once such dose level is deemed safe by the DLRT. Both the Medical Monitor and Principal investigator must deem it safe before a subject is considered for intra-subject dose escalation. The subject must be informed of the potential risks and benefits. Intra-subject dose escalation should only occur if the subject experienced ≤grade 1 toxicity during the previous course.

### Hepatotoxicity Stopping and Rechallenge Rules:

Guidelines for the management and monitoring of subjects with increased AST, ALT or alkaline phosphatase are outlined in Table 5.

TABLE 5
If the conditions for permanent discontinuation are met (below): Subject to be permanently discontinued
AST or ALT > 3 × ULN and INR > 1.5 × ULN (for subjects not on anticoagulation
therapy) in the presence of no important alternative causes for elevated AST/ALT values OR
AST or ALT > 3 × ULN and TBL > 2 × ULN in the presence of no
important alternative causes for elevated AST/ALT and/or TBL values
If conditions are not met: Exclude other causes^{a}
Upon failure to identify any other causes and sotorasib relation to
increase in LFTs cannot be excluded, proceed with guidelines below:

| | | Medical | |
|---|---|---|---|
| CTCAE Grade | Sotorasib Action | Management | Monitoring and Follow-up |
| Grade 2 AST or ALT | Continue | Consider steroids^{b} | Closely monitor liver function tests |
| and ALP ≤ 8 × ULN | | | |
| with no clinical | | | |
| symptoms consistent | | | |
| with hepatitis | | | |
| (right upper quadrant | | | |
| pain/tenderness, | | | |
| fever, nausea, | | | |
| vomiting, and | | | |
| jaundice) | | | |
| Grade 2 AST or ALT | First | Initiate steroids^{b} | Closely monitor liver function tests |
| with symptoms | Occurrence | | Await resolution to baseline or grade 1 and |
| Or | Withhold | | resolution or improvement of hepatitis |
| Grade 3 or 4 | | | symptoms |
| AST or ALT | | | Restart at 1 dose level reduction^{c, e} |
| Or | Second | Initiate steroids^{b} | Closely monitor liver function tests |
| 8 × ULN ALP^{d} | Occurrence | | Await resolution to baseline or grade 1 and |
| | Withhold | | resolution or improvement of hepatitis |
| | | | symptoms |
| | | | Resume at an additional 1 dose level |
| | | | reduction only with MEDICAL MONITOR |
| | | | approval^{c, e} |
| | Third | NOT APPLICABLE | |
| | Occurrence | | |
| | Permanently | | |
| | discontinue | | |
| | sotorasib | | |

ALP = alkaline phosphatase;
ALT = alanine aminotransferase;
AST = aspartate aminotransferase;
CTCAE = Common Terminology Criteria for Adverse Events;
INR = international normalized ratio;
LFT = liver function test;
TBL = total bilirubin;
ULN = upper limit of normal
^{a}If increase in AST/ALT is likely related to alternative agent, discontinue causative agent and await resolution to baseline or grade 1 prior to resuming sotorasib.
^{b}For example: Prednisone 1.0 to 2.0 mg/kg/day, dexamethasone equivalent, or methylprednisolone equivalent, followed by a taper. The taper may occur after restarting sotorasib.
^{c}Close monitoring at restart (e.g., daily LFTs × 2, then weekly × 4). Sotorasib dose may be increased after discussion with medical monitor.
^{d}There is no limit to the number of sotorasib re-challenges for isolated alkaline phosphatase elevations that resolve to baseline or grade 1.
^{e}Dose decrements below 240 mg are not allowable.

### Supportive Care Guidelines for Selected Expected Toxicities: RMC-4630:

In general, adverse events should be managed per institutional guidelines, if available, and should include evaluation for all potential underlying causes. Appropriate subspecialty consultation should be sought, as clinically indicated. Published guidelines are available for MEK and BRAF inhibitors (Welch and Corrie, 2015) and guidelines for selected toxicities are summarized below. Dose modification of RMC-4630 is described above.

### Management of Diarrhea:

Evaluate for other or concomitant causes, including medications (e.g., stool softeners, laxatives, and antacids), infection including Clostridium difficile, malabsorption/lactose intolerance, fecal impaction, and dietary supplements high in fiber.

For grade 1 or 2 diarrhea, dietary modifications should be considered including cessation of lactose-containing products, eat small meals, and ensure adequate hydration. Consumption of the banana, rice, apples, and toast (BRAT) diet may be helpful. In addition, loperamide or codeine may be used or alternatives in refractory cases such as octreotide and budesonide.

For >grade 2 diarrhea, consider hospitalization for fluid and electrolyte replacement and antibiotics, particularly if accompanied by fever or grade 3-4 neutropenia.

Guidelines for dose modification of RMC-4630 are discussed above. Consultation with a gastroenterologist should be considered, particularly for severe or refractory cases.

### Management of Peripheral or Facial/Periorbital Edema:

Observe for early signs of edema and investigate for potential other clinical causes including medications.

Follow institutional guidelines to manage edema including cessation of other medications known to be associated with the development of edema, if clinically indicated. Initiate treatment according to institutional guidelines (e.g., diuretics).

Guidelines for dose modification of RMC-4630 are discussed above.

### Management of Uncomplicated Thrombocytopenia:

These recommendations refer only to the management of uncomplicated thrombocytopenia, i.e., decrease in platelet counts without any accompanying clinical events of bleeding or significant bruising or other known risk factors for bleeding.

For grade 1-2 events (platelet level 50,000 to <100,000 [entry criteria]): continue treatment at current dose level and frequency and monitor closely.

For grade 3-4 events (platelet level <50,000): manage per institutional guidelines and interrupt dosing until patient recovers to grade 1 or better (platelet level >75,000). If recovery occurs, the investigator may resume dosing at the same dose level and frequency, if clinically appropriate. If grade 3-4 events recur again, hold dose until recovery to grade 1 or better and consider re-introducing study drug at reduced frequency, or withdrawing patient from study therapy.

For thrombocytopenia associated bleeding event or in patients with significant additional risk factors for bleeding, the general dose modification guidelines should be considered. The investigator may interrupt or discontinue study treatment as deemed medically appropriate.

### Management of Anemia:

Monitor closely and provide supportive care according to institutional standards. Interrupt study treatment as clinically appropriate. Dose interruption or discontinuation may not be required depending on baseline hemoglobin and transfusion history.

### Management of ILD/Pneumonitis:

Respiratory symptoms should be investigated with a chest X-ray or chest CT scan. Evaluate for possible infections and other causes of respiratory compromise including progressive pulmonary disease, cardiac dysfunction, or any environmental factors, etc.

Withhold RMC-4630 and any suspect combination drug if ILD/pneumonitis is suspected. If the diagnosis of ILD/pneumonitis is confirmed, permanently discontinue RMC-4630 and any suspect combination product.

Initiate treatment per Investigator judgement with corticosteroids and supportive care as per institutional guidelines.

### Management of Thromboembolic Events:

Thromboembolic events should be considered in the differential diagnosis for participants with shortness of breath, coughing, swelling, edema, or other potential cardiovascular adverse events. Evaluate with appropriate imaging study and treat according to institutional guidelines. Prophylaxis and/or hematology consultation may be considered for participants considered to have an increased risk of thromboembolic events.

### Treatment of Overdose:

Neither the effects of overdose of sotorasib nor an antidote to overdose are known.

For this study, any dose of RMC-4630≥1.5× than the intended dose per administration or 2 administrations at the intended dose within <24 hours will be considered an overdose. Ongoing evaluation of the clinical data and PK will be required to refine this definition as the data becomes available.

Revolution Medicines does not recommend specific treatment for an overdose. In the event of an overdose, the investigator should:
Contact the Medical Monitor immediately.
Closely monitor the patient for any adverse event/serious adverse event and laboratory abnormalities until RMC-4630 can no longer be detected systematically (at least 5 days).
Obtain a plasma sample for PK analysis within 1 to 2 days from the date of the last dose of study intervention if requested by the Medical Monitor (determined on a case-by-case basis).

### Prior and Concomitant Therapy

Prior therapies that were being taken from the time of initial diagnosis through the informed consent should be collected. For prior therapies being taken for the disease under study, collect therapy name, indication, dose, unit, frequency, start date and stop date. For all other prior therapies, collect therapy name, indication, dose, unit, frequency, route, start date, and stop date.

Throughout the study, investigators may prescribe any concomitant medications or treatments deemed necessary to provide adequate supportive care except for those listed in the exclusion criteria.

Concomitant administration of sotorasib with proton pump inhibitors (e.g., omeprazole) or H2-receptor antagonists (e.g., famotidine, etc.) decreased sotorasib concentrations, which may reduce sotorasib efficacy. Avoid use of PPIs or H2-receptor antagonists with sotorasib. If acid reducing therapy is considered medically necessary, administer antacids such as Maalox at least 10 hours before and/or 2 hours after sotorasib (i.e., take antacids the night before a morning sotorasib dose and wait 2 hours after sotorasib to take another antacid dose).

Breast cancer resistance protein (BCRP) substrates should be used with caution when co administered with sotorasib, which may increase the circulating concentrations of BCRP substrates.

As the absorption of RMC-4630 is pH sensitive antacids should be taken at a time point that is not proximal to the concomitant use of gastric pH lowering agent's administration (at least 3 to 4 hours before or after administration) to minimize any potential effect on drug absorption confounding the determination of the MTD and RP2D.

Concomitant therapies are to be collected from during screening and from cycle 1 day 1 through the end of the SFU period. For concomitant therapies being taken for the disease under study, collect therapy name, indication, dose, unit, frequency, start date and stop date. For all other concomitant therapies, collect therapy name, indication, dose, unit, frequency, route, start date, and stop date.

### Efficacy Assessments

Radiological Imaging Assessment: The extent of disease will be evaluated by contrast-enhanced MRI/CT according to RECIST v1.1. All radiological imaging will be performed as indicated in the Site Imaging Manual provided by the central imaging core laboratory. In order to reduce radiation exposure for subjects, the lowest dose possible should be utilized whenever possible.

The screening scans must be performed within 28 days prior to enrolment and will be used as baseline. Imaging performed as part of standard of care that falls within the screening window given for scans may be used for the baseline scan as long as it meets the scan requirements for screening. All subsequent scans will be performed in the same manner as at screening, with the same contrast, preferably on the same scanner. Radiological assessment must include CT of the chest, and contrast-enhanced CT or MRI of the abdomen and pelvis, as well as assessment of all other known sites of disease as detailed within the Site Imaging Manual.

The same imaging modality, MRI field strength and IV and oral contrast agents should be used at screening and for all subsequent assessments. Liver specific MRI contrast agents should not be used. To reduce potential safety concerns, macrocyclic gadolinium contrast agents are recommended per National Health Institute guidelines or follow local standards if more rigorous.

During treatment and follow-up, radiological imaging of the chest, abdomen, and pelvis, as well as all other known sites of disease, will be performed independent of treatment cycle every 6±1 weeks for the first 4 response assessments. After the 4 (6 week) response assessments, radiological imaging and tumor assessment will be performed every 12±1 weeks. Radiologic imaging and tumor assessment will be performed until disease progression, start of new anti-cancer treatment, death, withdrawal of consent or until end of study. Imaging may also be performed more frequently if clinically necessitated at the discretion of the managing physician. Radiographic response (CR and PR) requires confirmation by a repeat, consecutive assessment at least 4 weeks after the first documentation of response and may be delayed until the next scheduled scan to avoid unnecessary procedures.

All subjects must have MRI of the brain performed within 28 days prior to first dose of sotorasib and RMC-4630. Subsequently, MRI brain scans may be performed at any time if clinically indicated in the judgment of the managing physician. All brain scans on protocol are required to be MRI unless MRI is contraindicated, and then CT with contrast is acceptable.

Radiological imaging assessment at the end of treatment (EOT) visit, SFU, and LTFU should be performed only for subjects that discontinue treatment for a reason other than disease progression per RECIST v1.1 guidelines and only if they are due for a scan in that time interval. For subjects who discontinued study treatment without disease progression or the start of a subsequent anticancer treatment, tumor assessments will continue during LTFU every 12 weeks (±2 weeks) (or ad hoc to support analysis) until disease progression, start of subsequent anticancer treatment, death, withdrawal of consent, or for up to 3 years after the subject's last dose, whichever occurs first.

### Response Evaluation Criteria in Solid Tumors Version 1.1 (RECIST 1.1)

Determination of disease response for clinical management of subjects will be assessed at the clinical sites per RECIST v1.1. Scans will be submitted to a central imaging core laboratory for archival and if needed, independent response assessment utilizing RECIST v1.1 criteria. Exploratory imaging analyses may be performed centrally and may include tumor volumetrics, viable tumor measurements, tissue necrosis ratios, and lesion texture analysis (radiomics). Detailed information regarding submission of images to the central imaging core laboratory is found in the Site Imaging Manual.

### Definitions

### Measurable Lesions

Measurable Tumor Lesions—Non-nodal lesions with clear borders that can be accurately measured in at least 1 dimension with longest diameter ≥10 mm in computed tomography (CT)/magnetic resonance imaging (MRI) scan with slice thickness no greater than 5 mm. When slice thickness is greater than 5 mm, the minimum size of measurable lesion should be twice the slice thickness.

Nodal Lesions—Lymph nodes are to be considered pathologically enlarged and measurable, a lymph node must be ≥15 mm in short axis when assessed by CT/MRI (scan slice thickness recommended to be no greater than 5 mm). At baseline and in follow-up, only the short axis will be measured and followed.
Nodal size is normally reported as 2 dimensions in the axial plane. The smaller of these measures is the short axis (perpendicular to the longest axis).

Irradiated Lesions—Tumor lesions situated in a previously irradiated area, or in an area subjected to other loco-regional therapy, are not measurable unless there has been demonstrated progression in the lesion prior to enrollment.

### Non-Measurable Lesions

All other lesions, including small lesions (longest diameter <10 mm or pathological lymph nodes with ≥ 10 mm but to <15 mm short axis with CT scan slice thickness no greater than 5 mm) are considered non-measurable and characterized as non-target lesions.

Other examples of non-measurable lesions include:
Lesions with prior local treatment: tumor lesions situated in a previously irradiated area, or an area subject to other loco-regional therapy, should not be considered measurable unless there has been demonstrated progression in the lesion.
Biopsied lesions

Categorially, clusters of small lesions, bone lesions, inflammatory breast disease, and leptomeningeal disease are non-measurable.

### Methods of Measurement

Measurement of Lesions—The longest diameter of selected lesions should be measured in the plane in which the images were acquired (axial plane). All measurements should be taken and recorded in metric notation. All baseline evaluations should be performed as closely as possible to the beginning of treatment and not more than 4 weeks before study day 1.

Methods of Assessment—The same method of assessment and the same technique should be used to characterize each identified and reported lesion throughout the trial.

CT/MRI—Contrast-enhanced CT or MRI should be used to assess all lesions. Optimal visualization and measurement of metastasis in solid tumors requires consistent administration (dose and rate) of intravenous contrast as well as timing of scanning. Computed tomography and MRI should be performed with≤5 mm thick contiguous slices.

### Baseline Documentation of “Target” and “Non-Target” Lesions

Target Lesions—All measurable lesions up to a maximum of two (2) lesions per organ and five (5) lesions in total, representative of all involved organs should be identified as target lesions and recorded and measured at baseline.

Target lesions should be selected on the basis of their size (lesions with the longest diameter) and suitability for accurate repeated measurements.

Pathologic lymph nodes (with short axis ≥15 mm) may be identified as target lesions. All other pathological nodes (those with short axis ≥10 mm but <15 mm) should be considered non-target lesions.

A sum of the diameters (longest for non-nodal lesions, short axis for nodal lesions) for all target lesions will be calculated and reported as the baseline sum of diameters. The baseline sum of diameters will be used as reference by which to characterize objective tumor response.

Non-target Lesions-All other lesions (or sites of disease) including pathological lymph nodes should be identified as non-target lesions and should also be recorded at baseline. Measurements of these lesions are not required, and these lesions should be followed as “present,” “absent,” or “unequivocal progression” throughout the study. In addition, it is possible to record multiple non-target lesions involving the same organ as a single item on the case report form (CRF) (e.g., “multiple enlarged pelvic lymph nodes” or “multiple liver metastases”).

TABLE 6
EVALUATION OF TARGET LESIONS

| | |
|---|---|
| Complete Response (CR): | Disappearance of all target lesions. Any pathological lymph nodes |
| | (whether target or non-target) must have reduction in short axis |
| | to <10 mm. |
| Partial Response (PR): | At least a 30% decrease in the sum of the diameters of target lesions, |
| | taking as reference the baseline sum of diameters. |
| Progressive Disease (PD): | At least a relative 20% increase and an absolute increase of 5 mm in |
| | the sum of the diameters of target lesions, taking as reference the |
| | smallest sum on study, or the appearance of 1 or more new lesions. |
| Stable Disease (SD) | Neither sufficient shrinkage to qualify for PR nor sufficient increase |
| | to qualify for PD, taking as reference the smallest sum of diameters |
| | since the treatment started. |

TABLE 7
EVALUATION OF NON-TARGET LESIONS

| | |
|---|---|
| Complete Response (CR): | Disappearance of all non-target lesions and normalization of tumor |
| | marker levels. All lymph nodes must be non-pathological in size |
| | (<10 mm short axis). |
| Incomplete Response/Stable | Persistence of one or more non-target lesion(s) or/and maintenance |
| Disease (SD): | of tumor marker levels above the normal limits. |
| Progressive Disease (PD) | Unequivocal progression of existing non-target lesions and/or |
| | appearance of one or more new lesions.¹ |

¹To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal progression status.

### Evaluation of Overall Response

The best overall response is the best response recorded from the start of the study treatment until the end of treatment or disease progression/recurrence (taking as reference for PD the smallest measurements recorded since the treatment started).

In general, the subject's best response assignment will depend on the findings of both target and non-target disease and will also take into consideration the appearance of new lesions.

TABLE 8
TIME POINT RESPONSE: SUBJECTS WITH TARGET
(+/−NON-TARGET) DISEASE

| Target Lesions | Non-target Lesions | New Lesions | Overall Response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |

CR = complete response;
NE = Not evaluable;
PD = progressive disease;
PR = partial response;
SD = stable disease

TABLE 9
TIME POINT RESPONSE: SUBJECTS
WITH NON-TARGET DISEASE ONLY

| Non-target Lesions | New Lesions | Overall Response |
|---|---|---|
| CR | No | CR |
| Non-CR/non-PD | No | Non-CR/non-PD^{‡} |
| Not all evaluated | No | NE |
| Unequivocal PD | Yes or No | PD |
| Any | Yes | PD |

^{‡}“Non-CR/non-PD” is preferred over “SD” for non-target disease since SD is increasingly used as endpoint for assessment of efficacy in some trials so to assign this category when no lesions can be measured is not advised.
CR = complete response;
NE = Not evaluable;
PD = progressive disease;
SD = stable disease

TABLE 10
OVERALL RESPONSE: CONFIRMATION
OF COMPLETE RESPONSE (CR) AND
PARTIAL RESPONSE (PR) REQUIRED

| Overall | Overall | |
|---|---|---|
| Response | Response | |
| First | Second | |
| Time Point | Time Point | Best Overall Response |
| CR | CR | CR |
| CR | PR | SD, PD or PR^{†} |
| CR | SD | SD provided minimum criteria for SD |
| | | duration met, otherwise, PD |
| CR | PD | SD provided minimum criteria for SD |
| | | duration met, otherwise, PD |
| CR | NE | SD provided minimum criteria for SD |
| | | duration met, otherwise, NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise, NE |
| NE | NE | NE |

^{†}If a CR is truly met at first time point, then any disease at a subsequent time point, even disease meeting PR criteria relative to baseline, makes the disease PD at that point (since disease must have reappeared after CR). Best response would depend on whether minimum duration for SD was met. However, sometimes “CR” may be claimed when subsequent scans suggest small lesions were likely still present and in fact the subject had PR, not CR at the first time point. Under these circumstances, the original CR should be changed to PR and the best response is PR.
CR = complete response;
NE = Not evaluable;
PD = progressive disease;
PR = partial response;
SD = stable disease

### Special Notes on Response Assessment

Nodal lesions—Lymph nodes identified as target lesions should always have the actual short axis measurement recorded, even if the nodes regress to below 10 mm on study. In order to qualify for complete response (CR), each node must achieve a short axis <10 mm, NOT total disappearance. Nodal target lesion short axis measurements are added together with target lesion′ longest diameter measurements to create the sum of target lesion diameters for a particular assessment (time point).

Target lesions that become “too small to measure”—While on study, all lesions (nodal and non-nodal) recorded at baseline should have their measurements recorded at each subsequent evaluation. If a lesion becomes less than 5 mm, the accuracy of the measurement becomes reduced. Therefore, lesions less than 5 mm are considered as being “too small to measure,” and are not measured. With this designation, they are assigned a default measurement of 5 mm. No lesion measurement less than 5 mm should be recorded, unless a lesion totally disappears and “0” can be recorded for the measurement.

New lesions—The term “new lesion” always refers to the presence of a new finding that is definitely tumor. New findings that only may be tumor, but may be benign (infection, inflammation, etc.) are not selected as new lesions, until that time when the review is certain they represent tumor. If a new lesion is equivocal, for example because of its small size, continued therapy and follow-up evaluation will clarify if it represents truly new disease. If repeat scans confirm there is definitely a new lesion, then progression should be declared using the date of the initial scan. A lesion identified on a follow-up study in an anatomical location that was not scanned at baseline is considered a new lesion and will indicate disease progression, regardless of any response that may be seen in target or non-target lesions present from baseline.

Subjects with a global deterioration of health status requiring discontinuation of treatment without objective evidence of disease progression at that time should be classified as having “symptomatic deterioration.” Every effort should be made to document the objective progression with an additional imaging assessment even after discontinuation of treatment.

In some circumstances it may be difficult to distinguish residual disease from scar or normal tissue. When the evaluation of CR depends on this determination, it is recommended that the residual lesion be further investigated by fluorodeoxyglucose-positron emission tomography (FDG/PET) or PET/CT, or possibly fine needle aspirate/biopsy, to confirm the CR status.

### Confirmation Measurement/Duration of Response

Response Confirmation—In non-randomized trials where response is the primary endpoint, confirmation of partial response (PR) and CR is required to ensure responses identified are not the result of measurement error.

Duration of overall response—The duration of overall response is measured from the time measurement criteria are first met for CR/PR (whichever is first recorded) until the first date the recurrent or progressive disease is objectively documented.

Duration of Stable Disease—Stable disease (SD) is measured from the start of the treatment until the criteria for disease progression are met, taking as reference the smallest measurements recorded since the treatment started.

TABLE 11
ECOG PERFORMANCE AND NYHA CLASSIFICATION
ECOG Performance Status Scale

| Grade | Descriptions |
|---|---|
| 0 | Fully active, able to carry on all pre-disease performance without |
| | restriction. |
| 1 | Restricted in physically strenuous activity, but ambulatory and |
| | able to carry out work of a light or sedentary nature (e.g., light |
| | housework, office work). |
| 2 | Ambulatory and capable of all self-care, but unable to carry out |
| | any work activities. Up and about more than 50% of waking |
| | hours. |
| 3 | Capable of only limited self-care, confined to bed or chair more |
| | than 50% of waking hours. |
| 4 | Completely disabled. Cannot carry on any self-care. Totally |
| | confined to bed or chair. |
| 5 | Dead. |

Source: Oken et al, 1982; [0001]
ECOG = Eastern Cooperative Oncology Group

### New York Heart Association Functional Classification

Class I: No limitation of physical activity. Ordinary physical activity does not cause undue fatigue, palpitation or dyspnea.

Class II: Slight limitation of physical activity. Comfortable at rest, but ordinary physical activity results in fatigue, palpitation or dyspnea.

Class III: Marked limitation of physical activity. Comfortable at rest, but less than ordinary activity causes fatigue, palpitation or dyspnea.

Class IV: Unable to carry out any physical activity without discomfort. Symptoms of cardiac insufficiency may be present even at rest. If any physical activity is undertaken, discomfort is increased.

### Safety Assessments

Vital Signs: The following measurements must be performed: systolic/diastolic blood pressure, heart rate, respiratory rate, temperature, and pulse oximetry. Subject must be in a supine position in a rested and calm state for at least 5 minutes before blood pressure assessments are conducted.

If the subject is unable to be in the supine position, the subject should be in most recumbent position as possible. The position selected for a subject should be the same that is used throughout the study and documented on the vital sign CRF. The temperature location selected for a subject should be the same that is used throughout the study and documented on the vital signs CRF. Record all measurements on the vital signs CRF.

Electrocardiograms (ECGs): Subject must be in supine position in a rested and calm state for at least 5 minutes before ECG assessment is conducted. If the subject is unable to be in the supine position, the subject should be in most recumbent position as possible. Electrocardiograms should be performed in a standardized method, in triplicate, and run consecutively (all 3 ECGs should be completed within a total of 5 minutes from the start of the first to the completion of third), prior to blood draws or other invasive procedures. Each ECG must include the following measurements: QRS, QT, QTc, RR, and PR intervals.

Three baseline ECGs collected ≥30 minutes apart, with each baseline ECG in triplicate run consecutively (all 3 ECGs should be completed within a total of 5 minutes from the start of the first to the completion of the third) (total 9 ECGs).

Triplicate ECGs at time points after dosing.

Baseline is defined as screening. The PI will review all ECGs. Electrocardiograms will be transferred electronically to an ECG central reader for analysis per instructions. Once signed, the original ECG tracing will be retained with the subject's source documents. Standard ECG machines should be used for all study-related ECG requirements.

Vital Status: Vital status must be obtained for all subjects within the limits of local law. This includes subjects who may have discontinued study visits with or without withdrawing consent and should include interrogation of public databases, if necessary. If deceased, the date and reported cause of death should be obtained.

### Adverse Events

The adverse event grading scale to be used for this study will be the CTCAE v5.0. The investigator is responsible for ensuring that all serious adverse events observed by the investigator or reported by the subject that occur after the signing of the informed consent through the safety follow-up visit or 30 days after last day of dosing interval of investigational product(s)/protocol-required therapies, whichever is later, are reported using the Events CRF. The criteria for grade 4 in the CTCAE grading scale differs from the regulatory criteria for serious adverse events. If adverse events correspond to a grade 4 CTCAE toxicity grading scale criteria (e.g., laboratory abnormality reported as grade 4 without manifestation of life-threatening status), it will be left to the investigator's judgment to also report these abnormalities as serious adverse events. For any adverse event that applies to this situation, comprehensive documentation of the event's severity must be recorded in the subject medical records. If the investigator becomes aware of serious adverse events suspected to be related to investigational products after the protocol-required reporting period is complete, then these serious adverse events will be reported within 24 hours following the investigator's awareness of the event using the Events CRF. Since a study endpoint is overall survival, the investigator will also need to collect/report fatal serious adverse events (regardless of causality) within 24 hours following investigator's awareness of the event. After the End of Study, there is no requirement to actively monitor study subjects after the study has ended with regards to study subjects treated by the investigator. However, if the investigator becomes aware of serious adverse events suspected to be related to investigational products, then these serious adverse events will be reported within 24 hours following the investigator's awareness of the event. Serious adverse events reported outside of the protocol-required reporting period will be captured within the safety database as clinical trial cases and handled accordingly based on relationship to investigational product. If further safety related data is needed to fulfill any regulatory reporting requirements for a reportable event, then additional information may need to be collected from the subject's records after the subject ends the study.

Definition of Adverse Event—An adverse event is any untoward medical occurrence in a clinical study subject irrespective of a causal relationship with the study treatment. An adverse event can therefore be any unfavorable and unintended sign (including an abnormal laboratory finding), symptom, or disease (new or exacerbated) temporally associated with the use of a treatment, combination product, medical device or procedure. Treatment-emergent adverse events are defined elsewhere.

Events meeting the adverse event definition:

Any abnormal laboratory test results (hematology, clinical chemistry, or urinalysis) or other safety assessments (e.g., electrocardiogram, radiological scans, vital signs measurements), including those that worsen from baseline, that are considered clinically significant in the medical and scientific judgment of the investigator (i.e., not related to progression of underlying disease).

Exacerbation of a chronic or intermittent pre-existing condition including either an increase in frequency and/or intensity of the condition.

New conditions detected or diagnosed after study treatment administration even though it may have been present before the start of the study.

Signs, symptoms, or the clinical sequelae of a suspected drug-drug interaction.

Signs, symptoms, or the clinical sequelae of a suspected overdose of either study treatment or a concomitant medication. Overdose per se will not be reported as an adverse event/serious adverse event unless it is an intentional overdose taken with possible suicidal/self-harming intent. Such overdoses are to be reported regardless of sequelae.

For situations when an adverse event or serious adverse event is due to the advanced solid tumor, report all known signs and symptoms. Death due to disease progression in the absence of signs and symptoms should be reported as the primary tumor type (e.g., metastatic pancreatic cancer). Note: The term “disease progression” should not be used to describe the adverse event.

“Lack of efficacy” or “failure of expected pharmacological action” per se will not be reported as an adverse event or serious adverse event. Such instances will be captured in the efficacy assessments. However, the signs, symptoms, and/or clinical sequelae resulting from lack of efficacy will be reported as adverse event or serious adverse event if they fulfill the definition of an adverse event or serious adverse event.

Events not meeting the definition of adverse event:

Medical or surgical procedure (e.g., endoscopy, appendectomy): the condition that leads to the procedure is the adverse event.

Situations in which an untoward medical occurrence did not occur (social and/or convenience admission to a hospital).

Anticipated day-to-day fluctuations of pre-existing disease(s) or condition(s) present or detected at the start of the study that do not worsen.

### Definition of Serious Adverse Event—

Results in death (fatal).

Immediately life-threatening—The term “life-threatening” in the definition of “serious” refers to an event in which the subject was at risk of death at the time of the event. It does not refer to an event, which hypothetically might have caused death, if it were more severe.

Requires in-patient hospitalization or prolongation of existing hospitalization—In general, hospitalization signifies that the subject has been detained (usually involving at least an overnight stay) at the hospital or emergency ward for observation and/or treatment that would not have been appropriate in the physician's office or outpatient setting. Complications that occur during hospitalization are an adverse event. If a complication prolongs hospitalization or fulfills any other serious criteria, the event is serious. When in doubt as to whether “hospitalization” occurred or was necessary, the adverse event is to be considered serious. Hospitalization for elective treatment of a pre-existing condition that did not worsen from baseline is not considered an adverse event.

Results in persistent or significant disability/incapacity—The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.

Is a cognitive anomaly/birth defect.

Other medically important serious event-Medical or scientific judgment is to be exercised in deciding whether serious adverse event reporting is appropriate in other situations such as important medical events that may not be immediately life-threatening or result in death or hospitalization but may jeopardize the subject or may require medical or surgical intervention to prevent 1 of the other outcomes listed in the above definition. These events are typically to be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Evaluating Adverse Events and Serious Adverse Events

Assessment of Severity—The investigator will make an assessment of severity for each adverse event and serious adverse event reported during the study. The assessment of severity will be based on The Common Terminology Criteria for Adverse Events, version 5.0 which is incorporated by reference in its entirety.

Assessment of Causality—The investigator is obligated to assess the relationship between investigational product, protocol-required therapies, and/or study-mandated procedure and each occurrence of each adverse event/serious adverse event. Relatedness means that there are facts or reasons to support a relationship between investigational product and the event. The investigator will use clinical judgment to determine the relationship. Alternative causes, such as underlying disease(s), concomitant therapy, and other risk factors, as well as the temporal relationship of the event to study treatment administration will be considered and investigated. The investigator will also consult the investigator's Brochure and/or Product Information, for marketed products, in his/her assessment. For each adverse event/serious adverse event, the investigator must document in the medical notes that he/she has reviewed the adverse event/serious adverse event and has provided an assessment of causality. There may be situations in which a serious adverse event has occurred, and the investigator has minimal information to include in the initial report. However, it is very important that the investigator always make an assessment of causality for every event before the initial transmission of the serious adverse event data. The investigator may change his/her opinion of causality in light of follow-up information and send a serious adverse event follow-up report with the updated causality assessment. The causality assessment is 1 of the criteria used when determining regulatory reporting requirements.

### Preliminary Data (Data Cutoff-Apr. 11, 2022)

Patients with KRAS G12C-mutated NSCLC, CRC, or other solid tumors were treated with sotorasib (960 mg QD) and RMC-4630, with escalating dose levels of 100 mg, 140 mg, or 200 mg at days 1 and 2 or days 1 and 4 every 7 days. The primary endpoint was safety/tolerability. Secondary endpoints included objective response rate per RECIST 1.1 and pharmacokinetics.

Baseline characteristics of patients enrolled (27 patients as of Apr. 11, 2022 cutoff date) are shown in Table 12 below.

TABLE 12

| | | | Total | |
|---|---|---|---|---|
| | NSCLC | | (all tumors)* | |
| | (n = 11) | | (N = 27) | |
| Median age, years (range) | 64 | (51-78) | 63 | (43-78) |
| Male, n (%) | 4 | (36) | 15 | (56) |
| Smoking history, n (%)^{‡} | 11 | (100) | 19 | (70) |

ECOG performance score, n (%)

| 0 | 10 | (90) | 5 | (19) |
|---|---|---|---|---|
| 1^{†} | 0 | | 20 | (74) |
| Brain metastasis, n (%) | 7 | (64) | 13 | (48) |
| Liver metastasis, n (%) | 3 | (27) | 11 | (41) |
| Median lines of prior therapies, n (range) | 3 | (1-6) | 3 | (1-6) |
| Prior KRAS^{G12C} inhibitor, n (%)^{§} | 5 | (45) | 11 | (41) |
| Prior anti-PD-(L)1, n (%) | 10 | (91) | 15 | (56) |

*Total cohort includes patients with NSCLC (n = 11; 41%), CRC (n = 9; 33%), and other solid tumors (n = 7; 26%).
^{†}One patient's ECOG changed from 1 to 2 during screening, and one patient with missing entry.
^{‡}Includes former and current smoking history.
^{§}includes 8 patients treated with sotorasib and 3 patients treated with adagrasib.

Most common TRAEs are reported in Table 13 below, and no unexpected TRAEs were reported, based upon the safety profiles of each of sotorasib and RMC-4630. Results of TRAEs by dosing cohort are shown in Table 14. Pharmacokinetic analysis demonstrated that average sotorasib and RMC-4630 exposures were consistent with distributions observed in monotherapy studies, with no clinically meaningful drug-drug interactions noted. Efficacy of the combination therapy is shown in Table 15, where KRAS^{G12C} inhibitor-naïve patients showed improved efficacy over the average of all enrolled patients, with 100% of the KRAS^{G12C} inhibitor-naïve NSCLC patients exhibiting disease control.

TABLE 13

| | | | | | Sotorasib + RMC-4630 | |
|---|---|---|---|---|---|---|
| | Related to Sotorasib | | Related to RMC-4630 | | (N = 27)* | |
| Variable, n (%) | All Grades | Grade 3 | All Grades | Grade 3 | All Grades | Grade 3 |
| Total TRAE | 15 (56) | 6 (22) | 17 (63) | 6 (22) | 17 (63) | 6 (22) |
| Edema ^{†} | 7 (26) | 0 | 6 (22) | 0 | 8 (30) | 0 |
| Diarrhea | 7 (26) | 2 (7) | 5 (19) | 2 (7) | 7 (26) | 2 (7) |
| Dry mouth | 3 (11) | 0 | 2 (7) | 0 | 3 (11) | 0 |
| Fatigue | 3 (11) | 0 | 3 (11) | 0 | 3 (11) | 0 |
| AST increased | 1 (4) | 1 (4) | 2 (7) | 1 (4) | 2 (7) | 1 (4) |
| Ascites | 1 (4) | 1 (4) | 1 (4) | 1 (4) | 1 (4) | 1 (4) |
| Colitis | 1 (4) | 1 (4) | 1 (4) | 1 (4) | 1 (4) | 1 (4) |
| Dyspnea | 1 (4) | 1 (4) | 1 (4) | 1 (4) | 1 (4) | 1 (4) |
| Hypertension | 0 | 0 | 1 (4) | 1 (4) | 1 (4) | 1 (4) |
| Pleural effusion | 0 | 0 | 1 (4) | 1 (4) | 1 (4) | 1 (4) |

*Related to either study drug across all doses. Includes TRAEs with >10% patient incidence across all grades. No Grade 4 or 5 TRAEs.
^{†} Includes general, peripheral, periorbital, and facial edema.

TABLE 14

| | Sotorasib 960 mg + | | Sotorasib 960 mg + | Sotorasib 960 mg + | |
|---|---|---|---|---|---|
| | RMC-4630 100 mg | | RMC-4630 140 mg | RMC-4630 200 mg | |
| Variable*, n (%) | (N = 6) | | (N = 10) | (N = 11) | |
| TRAE any grade | 6 | (100) | 4 (40) | 7 | (64) |
| Grade 3 | 1 | (17) | 1 (10) | 4 | (36) |
| TRAE leading to sotorasib dose | 1 | (17) | 1 (10) | 3 | (27) |
| modification^{†} | | | | | |
| TRAE leading to RMC-4630 | 2 | (33) | 1 (10) | 4 | (36) |
| dose modification^{†} | | | | | |
| TRAE leading to sotorasib | 1 | (17) | 0 | 0 | |
| discontinuation^{‡} | | | | | |
| TRAE leading to RMC-4630 | 1 | (17) | 1 (10) | 1 | (9) |

| | | | |
|---|---|---|---|
| discontinuation^{‡§} | | | |
| Number of patients with a dose | 1 | 1 | 0 |
| limiting toxicity^{¶} | | | |

*Related to either study drug.
^{†}Patients dose reduced or interrupted both drugs for increased ALT, diarrhea, pleural effusion, colitis, fatigue (1 each); sotorasib only for diarrhea, increased AST, dry mouth, dyspnea (1 each); RMC-4630 only for peripheral edema, ascites, hypertension, constipation, liver function test increased (1 each).
^{‡}One patient had increases in AST leading to discontinuation of both RMC-4630 and sotorasib.
^{§}One patient had diarrhea and one patient had ascites leading to RMC-4630 discontinuation.
^{¶}Patient experienced grade 3 AST elevation at 100 mg RMC-4630 and grade 3 ascites at 140 mg RMC-4630.

TABLE 15

| | NSCLC | | | |
|---|---|---|---|---|
| | All enrolled | | KRAS^{G12C} inhibitor-naive | |
| | (n = 11) | | (n = 6) | |
| ORR, % (95% CI) | 27 | (6, 61) | 50 | (12, 88) |
| Best overall response, n (%) | | | | |
| Partial response, confirmed | 3 | (27) | 3 | (50) |
| Stable disease | 4 | (36) | 3 | (50) |
| Progressive disease | 4 | (36) | 0 | |
| Disease control rate, n (%) | 7 | (64) | 6 | (100) |

ORR—objective response rate

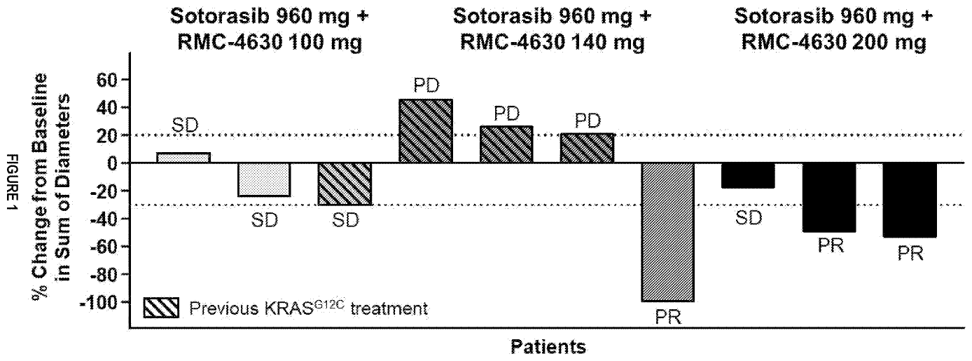

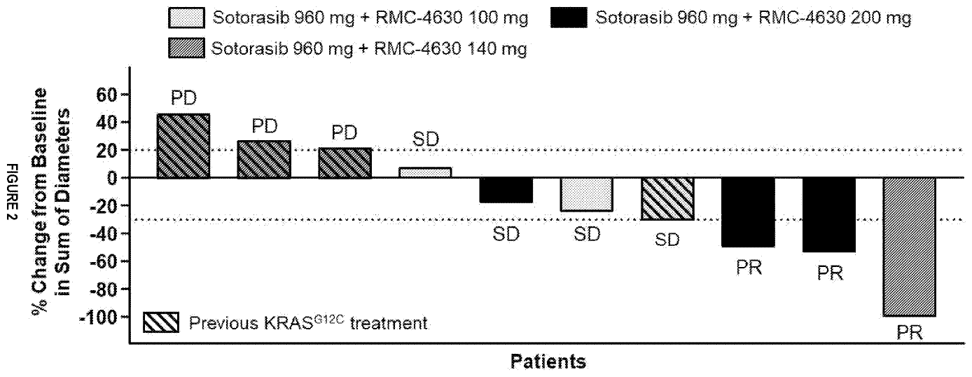

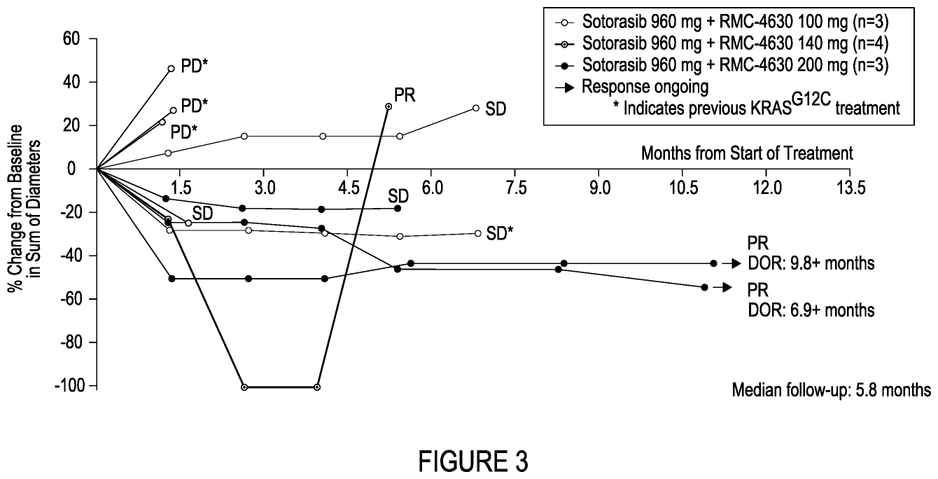

Tumor response in non-small cell lung cancer is shown in FIG. 1, which shows the % change from baseline in sum of diameters for 10 patients taking 960 mg sotorasib and different doses of RMC-4630 (100 mg, 140 mg or 200 mg). FIG. 2 shows the same data, but arranged from progressive disease to partial response. At the highest RMC-4630 dose (200 mg) the responders included three of four patients who were KRAS^{G12C}-inhibitor naiver. Treatment duration of patients with NSCLC is shown in FIG. 3, showing the response of each patient and their dose of RMC-4630. Two of three patients with partial response had an ongoing response at data cutoff. One patient whose NSCLC progressed on prior sotorasib treatment achieved an unconfirmed partial response at 24 weeks with progressive disease at 30 weeks.

The combination of sotorasib with RMC-4630 was safe and tolerable in patients with KRAS G12C-mutated solid tumors. Promising clinical activity was observed in KRAS G12C-mutated NSCLC patients, most notably in those KRAS^{G12C} inhibitor-naïve.

### REFERENCES

Albert et al., Nat. Methods 2007; 4:903-905.
The American Association for Cancer Research [AACR] Project GENIE Consortium, 2017 https://www.aacr.org/RESEARCH/RESEARCH/PAGES/AACR-PROJECT-GENIE.ASPX.
Beers and Nederlof, Breast Cancer Res. 2006; 8 (3): 210.
Bertone et al., Genome Res 2006; 16 (2): 271-281.
Canon et al., Nature. 2019; 575 (7781): 217-223.
Cerami et al., Cancer Discov. 2012, 2 (5), 401.
Chung et al., Genome Res. 2004; 14 (1): 188-196.
Cully and Downward, Cell. 2008; 133:1292.
Dalma-Weiszhausz et al., Methods Enzymol. 2006; 410:3-28.
DeRisi et al., Nat. Genet. 1996; 14:457-460.
Eisenhauer et al., Eur J Cancer, 2009; 45 (2): 228-247.
www.fda.gov/drugs/drug-interactions-labeling/drug-development-and-drug-interactions-table-substrates-inhibitors-and-inducers, accessed May 2021.
Flockhart, Drug Interactions: Cytochrome P450 Drug Interaction Table. Indiana University School of Medicine (2007), www.drug-interactions.medicine.iu.edu, accessed May 2021.
Forshew et al., Sci Transl Med; 2012; 4: 136ra68.
Gao et al., Science Signaling 2013, 6(269), pl1.
Haber and Velculescu, Cancer Discov., 2014; 4:650-61.
Hong et al., N Engl J Med. 2020; 383(13): 1207-1217.
Hughes et al., Nat. Biotechnol. 2001; 19(4): 342-347.
Irizarry, Nucleic Acids Res 2003, 31:e15.
Janes et al., Cell. 2018; 172 (3): 578-589.
Jasmine et al., PLoS One 2012; 7 (2):e31968.
Ji et al., Clin Trials 2010; 7 (6): 653-663.
Jones et al., Br J Cancer 2017; 116 (7): 923-929.
Kim et al., Carcinogenesis 2006; 27 (3): 392-404.
Kinde et al., Proc Natl Acad Sci USA; 2011, 108:9530-5.
Kumar et al., J. Pharm. Bioallied Sci. 2012; 4 (1): 21-26.
Laere et al., Methods Mol. Biol. 2009; 512:71-98.
Lanman et al., J. Med. Chem. 2020, 63, 52.
Lin et al., BMC Genomics 2010; 11:712.
Lin et al., Lancet Oncol. 2015; 16:e270-278.
Lito et al., Science 2016; 351 (6273): 604-608.
Liu et al., Biosens Bioelectron 2017; 92:596-601.
Lodes et al., PLoS One 2009; 4 (7):e6229.
LUMAKRAS® US Prescribing Information, Amgen Inc., Thousand Oaks, California, 91320 (revision May 2021).
Mackay et al., Oncogene 2003; 22:2680-2688.
Mao et al., Curr. Genomics 2007; 8 (4): 219-228.
McDonald et al., Cell. 2017; 170 (3): 577-592.
Michels et al., Genet. Med. 2007; 9:574-584.
Mockler and Ecker, Genomics 2005; 85 (1): 1-15.
National Cancer Institute Common Terminology Criteria for Adverse Events v5.0 (NCI CTCAE) published Nov. 27, 2017.
Oken et al., 1 Am J Clin Oncol. 1982; 5 (6): 649-655.
Ostrem et al., Nature. 2013; 503 (7477): 548-551.
Ostrem and Shokat, Nat Rev Drug Discov. 2016; 15 (11): 771-785.
Patricelli et al., Cancer Discov. 2016; 6 (3): 316-329.
Pinkel et al., Nat. Genetics 2005; 37: S11-S17.
Ruess et al., Nat Med 2018; 24 (7): 954-960.
Simanshu et al., Cell. 2017; 170 (1): 17-33.
Thomas et al. Genome Res. 2005; 15 (12): 1831-1837.
Thompson et al., PLOS ONE, 2012; 7:e31597.
Wang et al., Cancer Genet 2012; 205 (7-8): 341-55.
Wei et al., Nucleic Acids Res 2008; 36 (9): 2926-2938.
Welch and Corrie, Nat Rev Clin Oncol. 2015; 7 (3): 153-162.
Wen et al., J Clin Oncol. 2010; 28:1963-1972.
Xie et al., Front Pharmacol.; 2017; 8:823.
Zhou et al., Med Oncol., 2016, 33 (4): 32.
Zubrod et al., Chronic Disease, 1960; 11:7-33.

What is claimed is:

1. A method of treating cancer in a patient, wherein the patient has a KRAS G12C mutation, the method comprising
administering to the patient a therapeutically effective combination of sotorasib and {6-[(2-amino-3-chloropyridin-4-yl)sulfanyl]-3-[(3S,4S)-4-amino-3-methyl-2-oxa-8-azaspiro[4.5]decan-8-yl]-5-methylpyrazin-2-yl}methanol (RMC-4630).

2. The method of claim 1, comprising administering 960 mg sotorasib daily.

3. The method of claim 1 or 2, comprising administering sotorasib orally once daily.

4. The method of any one of claims 1 to 3, comprising administering 50 to 200 mg RMC-4630, 100 mg RMC-4630, 140 mg RMC-4630, 200 mg RMC-4630, or 50 mg RMC-4630.

5. The method of any one of claims 1 to 4, wherein RMC-4630 is administered orally on days 1 and 2 of each 7 days, or on days 1 and 4 of each 7 days.

6. The method of any one of claims 1 to 5, wherein RMC-4630 is administered to the patient in a fasted state.

7. The method of any one of claims 1 to 6, wherein RMC-4630 is administered to the patient after a 2 hour fast.

8. The method of any one of claims 1 to 7, wherein the patient fasts for 1 hour after the administration of RMC-4630.

9. The method of any one of claims 1 to 8, wherein sotorasib and RMC-4630 are administered for a treatment cycle, and the treatment cycle is 21 days.

10. The method of claim 9, wherein the patient undergoes at least 3 treatment cycles, or at least 8 treatment cycles.

11. The method of any one of claims 1 to 10, wherein the cancer is a solid tumor.

12. The method of any one of claims 1 to 11, wherein the cancer is non-small cell lung cancer, small bowel cancer, appendiceal cancer, colorectal cancer, cancer of unknown primary, endometrial cancer, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, ampullary cancer, gastric cancer, sinonasal cancer, or bile duct cancer.

13. The method of any one of claims 1 to 12, wherein the cancer is non-small cell lung cancer, small bowel cancer, appendiceal cancer, colorectal cancer, cancer of unknown primary, endometrial cancer, pancreatic cancer, melanoma, ampullary cancer, gastric cancer, sinonasal cancer, or bile duct cancer.

14. The method of any one of claims 1 to 13, wherein the cancer is non-small cell lung cancer.

15. The method of claim 14, wherein the non-small cell lung cancer is locally advanced or metastatic.

16. The method of any one of claims 1 to 13, wherein the cancer is colorectal cancer.

17. The method of any one of claims 1 to 12, wherein the cancer is a solid tumor other than colorectal cancer or non-small cell lung cancer.

18. The method of any one of claims 1 to 17, wherein the patient did not previously receive a prior line of anti-cancer therapy.

19. The method of any one of claims 1 to 17, wherein the patient received at least one prior line of anti-cancer therapy.

20. The method of any one of claims 1 to 19, wherein the patient did not previously receive an anti-PD1 or anti-PD-L1 immunotherapy.

21. The method of any one of claims 1 to 17 and 19, wherein the patient previously received an anti-PD1 or anti-PD-L1 immunotherapy.

22. The method of any one of claims 1 to 17 and 19 to 21, wherein the patient previously received platinum-based chemotherapy.

23. The method of any one of claims 1 to 22, wherein the patient did not previously receive (i) anti-PD1 or anti-PDL1 immunotherapy or (ii) platinum-based chemotherapy.

24. The method of any one of claims 1 to 17, 19, 21, and 22, wherein the patient previously received (i) anti-PD1 or anti-PD-L1 immunotherapy and (ii) platinum-based chemotherapy.

25. The method of any one of claims 1 to 17 and 19 to 24, wherein the patient (i) has a mutation in EGFR, ALK, or ROS1, and (ii) previously received an EGFR, ALK, or ROS1 targeted therapy.

26. The method of claim 25, wherein the patient progressed on the EGFR, ALK or ROS1 targeted therapy.

27. The method of any one of claims 1 to 11, wherein the cancer is non-small cell lung cancer, and
(1) (a) the patient previously received (i) anti-PD1 or anti-PD-L1 immunotherapy or (ii) platinum-based chemotherapy; or (b) the patient previously received (i) anti-PD1 or anti-PD-L1 therapy and (ii) platinum-based chemotherapy; and
(2) the patient optionally has a mutation in EGFR, ALK, or ROS1 and previously received an EGFR, ALK or ROS1 targeted therapy.

28. The method of any one of claims 1 to 11, wherein the cancer is colorectal cancer and
(1) the patient received at least one prior systemic regime of therapy for advanced or metastatic colorectal cancer comprising fluoropyrimidine, oxaliplatin, or irinotecan-based therapy; and
(2) the patient has a MSI-H cancer and received therapy with nivolumab or pembrolizumab, if the patient was clinically able to receive checkpoint inhibitors (e.g., PD1 or PD-L1 inhibitors).

29. The method of any one of claims 1 to 28, wherein the patient did not receive a prior therapy with a KRAS^{G12C} inhibitor.

30. The method of any one of claims 1 to 17 and 19 to 28, wherein the patient received a prior therapy with a KRAS^{G12C} inhibitor.

31. The method of claim 30, wherein the patient progressed on the prior therapy with the KRAS^{G12C} inhibitor.

32. The method of any one of claims 1 to 31, wherein the patient has an Eastern Cooperative Oncology Group (ECOG) performance status of less than or equal to 1.

33. The method of any one of claims 1 to 32, wherein the patient does not have a primary brain tumor.

34. The method of any one of claims 1 to 33, wherein the patient does not have spinal cord compression or untreated or symptomatic brain metastases or leptomeningeal disease from nonbrain tumors.

35. The method of any one of claims 1 to 34, wherein
(i) the patient
had brain metastases resected, or
received whole brain radiation therapy ending at least 4 weeks prior to start of first treatment cycle, or
received stereotactic radiosurgery ending at least 2 weeks prior to first treatment cycle, and
(ii) the patient
exhibits residual neurological symptoms of grade 2 or less, and
receives a stable dose of dexamethasone, if applicable; and
has an magnetic resonance imaging (MRI) performed within 30 days prior to first treatment cycle shows no new or enlarging lesions appearing.

36. The method of any one of claims 1 to 35, wherein the patient does not have either 160 mmHg or greater systolic blood pressure or 100 mmHg or greater diastolic blood pressure.

37. The method of any one of claims 1 to 36, wherein the patient is in further need of treatment with a CYP3A4 substrate.

38. The method of claim 37, wherein the patient is not administered a CYP3A4 substrate during any treatment cycle or within 14 days or 5 half-lives of the CYP3A4 substrate or its major active metabolite, whichever is longer, before starting administration of sotorasib and RMC-4630.

39. The method of claim 37 or 38, wherein the CYP3A4 substrate is a CYP3A4 substrate with a narrow therapeutic index.

40. The method of any one of claims 1 to 39, wherein the patient is in further need of treatment with a P-glycoprotein (P-gp) substrate.

41. The method of claim 40, wherein the patient is not administered a P-gp substrate during any treatment cycle or within 14 days or 5 half-lives of the P-gp substrate or its major active metabolite, whichever is longer, before starting administration of sotorasib and RMC-4630.

42. The method of any one of claims 1 to 41, wherein the patient is in further need of treatment with a strong CYP3A4 inducer, and optionally is not administered a strong CYP3A4 inducer during any treatment cycle or within 14 days or 5 half-lives of the strong CYP3A4 inducer, whichever is longer, before starting administration of sotorasib and RMC-4630.

43. The method of any one of claims 1 to 42, wherein the patient has not received prior SHP2 inhibitor therapy.

44. The method of any one of claims 1 to 43, wherein the patient is in further need of treatment with a proton pump inhibitor (PPI) or a H2 receptor antagonist (H2RA), and optionally is not administered a PPI or a H2RA during any treatment cycle or within three days of starting administration of sotorasib and RMC-4630.

45. The method of any one of claims 1 to 44, wherein the patient exhibits at least a stable disease (SD), as measured by RECIST 1.1 protocol, after at least 3, 6, or 8 months, or at least a partial response (PR), as measured by RECIST 1.1 protocol, after at least 3, 6, or 8 months.

46. The method of any one of claims 1 to 45, wherein the patient exhibits a progression free survival (PFS) of at least 3 months.
